(19) United States
(12) Patent Application Publication
(10) Pub. No.: US 2025/0273296 A1
(43) Pub. Date: Aug. 28, 2025

(54) METHOD OF DETECTING CANCER DNA IN A SAMPLE

(71) Applicant: INIVATA LTD., Cambridge (GB)

(72) Inventors: Artur RUUGE, Cambridge (GB); Warren EMMETT, Cambridge (GB); Giovanni MARSICO, Cambridge (GB); Tim FORSHEW, Stevanage (GB)

(21) Appl. No.: 19/101,565

(22) PCT Filed: Aug. 17, 2023
(86) PCT No.: PCT/IB2023/058239

§ 371 Date: Feb. 5, 2025

(30) Foreign Application Priority Data
Aug. 19, 2022 (GB) 2212094.3

(51) Int. Cl. G16B 20/20; C12Q 1/686; C12Q 1/6869; C12Q 1/6886; G16B 20/10; G16B 40/20; G16H 15/00; G16H 50/20; G16H 50/70

(52) U.S. Cl. CPC G16B 20/20; C12Q 1/686; C12Q 1/6869; C12Q 1/6886; G16B 20/10; G16B 40/20; G16H 15/00; G16H 50/20; G16H 50/70

(57) ABSTRACT

In one embodiment, the method may comprise enriching the test sample for a plurality of target regions, wherein the plurality of target regions comprises a first target region having a first class and a second target region having a second class. The plurality of target regions may be measured and for each of the first target region and second target region, the measurements that support the class of the target region may be compared to an error model that models the probability of observing that class of target region in DNA that does not contain that class of target region. These comparisons may then be combined for at least the first target region and the second target region. Cancer DNA may then be identified in the test sample based on the combined comparisons.

### CROSS-REFERENCING

This application claims the benefit of United Kingdom patent application serial number GB2212094.3, filed on Aug. 19, 2022, which application is incorporated by reference herein for all purposes.

### FIELD

The disclosure generally relates to the field of liquid biopsy, such as diagnosing the presence of cancer in blood or other fluid samples from patients.

### BACKGROUND

Detection and monitoring of circulating tumor DNA (ctDNA) is rapidly becoming a diagnostic, prognostic, and predictive tool in cancer patient care. Often, after treatment for cancer, a small number of cancer cells may remain within a patient who appears to be in remission. These residual cells are often called “minimal residual disease” (MRD) or residual disease. These residual cells will ultimately be the cause of relapse in many cancers. It is critical to determine the likelihood of a patient having disease recurrence and relapsing following initial treatment so that those most likely to need additional treatment can receive that additional treatment, while those that don't need additional treatment are spared, thereby reducing harm to the patient and decreasing the cost of treatment. As such, effective methods for the detecting of minimal residual disease are highly desirable. It is also critical to have sensitive methods that detect risks of cancer recurrence earlier than current methods (e.g., which are usually done by imaging or clinical analysis).

MRD has been successfully detected in some hematological malignancies because relatively large amounts of DNA can be analyzed and the frequency of common tumor specific fusions can be measured in a straightforward way. There is now strong evidence that MRD can be detected for many solid tumors by assessing cell free DNA (cfDNA) for circulating tumor DNA (ctDNA). The problem with detecting minimal residual disease in cfDNA, however, is that many of the tests used to detect sequence variations in a sample are not sufficiently sensitive. Many of today's molecular tests are done by sequencing cfDNA for a panel of known genes. The problem with detecting minimal residual disease by sequencing cfDNA is that the amount of tumor DNA in cell-free DNA is often well below the limit of detection of such methods. Specifically, the frequency at which an individual tumor sequence variation is expected to occur in the cfDNA of patients that have minimal residual disease is typically well below the frequency at which sequencing artefacts are generated by PCR errors, base mis-calls, and/or DNA damage. This problem is compounded by the fact that, in some cases, the level of tumor DNA may be so low that, on average, there is less than a single copy of each mutation being assessed in the cfDNA sample being analyzed. In addition, relatively small amounts of mutant DNA derived from white blood cells that have lysed in the bloodstream can lead to erroneous results. Thus, detection of minimal residual disease by sequencing-based approaches has remained challenging.

Assays for detecting MRD can employ a variety of approaches, including sequencing a patient's tumor tissue to identify tumor-specific genetic variants. These variants can include single nucleotide variants, small insertions and deletions, doublet base substitutions, and larger structural changes. Identifying these tumor-specific variants in a patient's cfDNA sample should, in theory, be indicative of MRD. However, as the number of tumor-specific variants in an assay grows, the potential for a false positive result may be increased. Additionally, different kinds of variants may be more or less likely to produce a false positive result. Accordingly, there is a need for improvement in ctDNA detection.

### SUMMARY

Described herein are methods for detecting cancer DNA in a test sample of DNA from a patient. In some embodiments, the method may comprise enriching the test sample for a plurality of target regions, wherein the plurality of target regions comprises a first target region having a first class and a second target region having a second class. The plurality of target regions may be measured and for each of the first target region and second target region, the measurements that support the class of the target region may be compared to an error model that models the probability of observing that class of target region in DNA that does not contain that class of target region. These comparisons may then be combined for at least the first target region and the second target region. Cancer DNA may then be identified in the test sample based on the combined comparisons.

The methods described herein, in one example, are derived from the realization that the problem of low sensitivity when determining the presence of cancer DNA in a patient test sample can be solved by combining evidence from multiple target regions having different kinds of genetic variations and different numbers of genetic variations. Observations from each target region provide some evidence which may be combined to support a high-confidence conclusion that the test sample contains cancer DNA and thus the patient has cancer, or residual disease. Further, methods described herein can combine evidence from multiple classes of target regions, such as target regions containing single nucleotide variations, multiple nucleotide variants (such as doublet and triplet base substitutions), short insertions or deletions, copy number variants, structural variants (SVs), multiple genetic variants, and multiple phased variants (i.e., wherein the target region has two or more variants all on the same chromosome within the target region). Each of these classes may provide differing levels of support or confidence as to the presence (or absence) of cancer, and therefore must be combined in a principled manner, as further described herein.

These and other advantages may become apparent in view of the following discussion.

### BRIEF DESCRIPTION OF THE DRAWINGS

One of ordinary skill in the art will understand that the drawings, described below, are for illustration purposes only. The drawings are not intended to limit the scope of the present teachings in any way.

FIG. 1 is a flow chart depicting an embodiment of a method of detecting cancer DNA in a test sample of DNA from a patient

FIG. 2A is an illustration depicting an embodiment of an enhanced tagged amplicon sequencing (eTAm-Seq™) approach in which target regions are amplified by a polymerase chain reaction (PCR).

FIG. 2B depicts several exemplary classes of target regions.

FIG. 3 is an illustration depicting an exemplary assay of target regions containing genetic variations according to an embodiment of the disclosure.

FIGS. 4A-B illustrate examples of error probability distributions according to an embodiment of the disclosure. In the model shown in FIG. 4A, the data corresponding to low frequency high signal events are hatched. Two models are shown in FIG. 4B, one for background noise and another for DNA damage. “VAF” refers to variant allele fraction. Such models may be obtained from DNA that does not contain the genetic variation and they indicate the probability of different variant allele fractions in this non-cancerous DNA (or the number of variant reads over the total reads).

FIG. 5 is a block diagram of an illustrative computer system that may be used in implementing some embodiments of the technology described herein.

FIGS. 6A-6B depict an embodiment of an assay as described herein and illustrate some of the difficulties in detecting cancer DNA by methods in which individual target regions are scored for whether they contain a particular genetic variant or not.

FIG. 7 schematically illustrates some of the principles of an embodiment of the present method.

FIG. 8 shows how the fraction of cancer DNA can be calculated by comparing real dilution data to a mathematical model.

FIG. 9 is a figure adapted from Kurtz et al (Nat Biotechnol 2021 39: 1-11) in which the authors showed that the genome has a small number of phased variants (part b).

FIG. 10 is a figure adapted from Li et al (Nature 2020 578, 112-121) that shows that the number of structural variants and the range of types of structural variants in different types of cancer. Some types of cancers often have large numbers of structural variants such as breast cancer and certain sarcoma whilst others, such as CLL, typically have low numbers.

### DEFINITIONS

Unless otherwise defined, all technical and scientific terms used herein have the same meaning as commonly understood by one of ordinary skill in the art to which the disclosure belongs. Still, certain elements are defined for the sake of clarity and ease of reference.

Terms and symbols of nucleic acid chemistry, biochemistry, genetics, and molecular biology used herein follow those of standard treatises and texts in the field, e.g. Kornberg and Baker, DNA Replication, Second Edition (W.H. Freeman, New York, 1992); Lehninger, Biochemistry, Eighth Edition (Worth Publishers, New York, 2021); Strachan and Read, Human Molecular Genetics, Fifth Edition (Wiley-Liss, New York, 2018); Eckstein, editor, Oligonucleotides and Analogs: A Practical Approach (Oxford University Press, New York, 1992); Gait, editor, Oligonucleotide Synthesis: A Practical Approach (IRL Press, Oxford, 1984); (the contents of which are incorporated by reference in their entireties) and the like.

As used herein, depending on the context the term “calling” can mean indicating whether a particular genetic variation is present in a sequence, whether a sample contains a genetic variation, or whether a sample contains cancer DNA.

If two nucleic acids are “complementary,” they hybridize with one another under high stringency conditions. The term “perfectly complementary” describes a duplex in which each base of one of the nucleic acids base pairs with a complementary nucleotide in the other nucleic acid. In many cases, two sequences that are complementary have at least 10, e.g., at least 12 or 15 nucleotides of complementarity.

As used herein, the term “detecting recurrence” refers to detecting the recurrence of a tumor through the identification of cancer DNA. In this context, the term “early detection” refers to the detection of mutant DNA before cancer recurrence can be reliably detected through conventional standard-of-care/surveillance monitoring methods such as radiological imaging etc. This may be achieved for example by monitoring serially collected blood samples at a plurality of time points for the presence of ctDNA in cfDNA, as described below.

The terms “determining”, “measuring”, “evaluating”, “assessing” and “assaying” are used interchangeably and include quantitative and qualitative determinations. Assessing may be relative or absolute.

The term “genetic variation”, as used herein, refers to a variation (e.g., a nucleotide substitution, an indel or a rearrangement) that is present or deemed as being likely to be present in a test sample. A genetic variation can be from any source. For example, a genetic variation can be generated by a mutation (e.g., a somatic mutation), or it can be germline, such as mutations derived from reproductive cells that become incorporated into the DNA of every cell in the body. If a sequence variation is called as a genetic variation, the call indicates that the sample likely contains the variation; but, in some cases a “call” can be incorrect. In many cases, the term “genetic variation” can be replaced by the term “mutation”. For example, if a method is being used to detect sequence variations that are associated with cancer or other diseases that are caused by mutations, then “genetic variation” can be replaced by the term “mutation”.

As used herein, the term “minimal residual disease” (MRD), refers to the presence of cancer cells following a treatment with curative intent. MRD may also be referred to as “molecular residual disease” or “residual disease” in some publications.

The term “nucleic acid”, “oligonucleotide”, and “polynucleotide” are used interchangeably herein to describe a polymer of any length, e.g., greater than about 2 bases, greater than about 10 bases, greater than about 100 bases, greater than about 500 bases, greater than 1000 bases, greater than 10,000 bases, greater than 100,000 bases, greater than about 1,000,000, up to about 1010 or more bases composed of nucleotides, e.g., deoxyribonucleotides or ribonucleotides.

The terms “plurality”, “population”, and “collection” are used interchangeably to refer to something that contains at least 2 members. In certain cases, a plurality, population, or collection may have at least 5, at least 10, at least 100, at least 1,000, at least 10,000, at least 100,000, at least 10⁶, at least 10⁷, at least 10⁸ or at least 10⁹ or more members.

The term “reference sequence”, as used herein, is a reference sequence from a reference genome or sequence from a sample of a patient not anticipated to contain somatic variants such as a buccal swab. A reference sequence corresponds to a sequence (e.g., a target sequence) that contains or may be suspected of containing a sequence variation, hence enabling the existence (or not) of a sequence variation to be determined by comparing the sequence (e.g., the target sequence) that contains or may be suspected of containing a sequence variation to the reference sequence. A reference sequence differs from the sequence (e.g. a target sequence) that contains or may be suspected of containing the sequence variation only in the sequence variation itself, since the reference sequence and the sequence (e.g. a target sequence) that contains or may be suspected of containing a sequence variation originates from the same genomic location.

The term “reference genome”, as used herein, may refer to a single genome, a collection of genomes, or a consensus genome. The reference genome may be from one or more publicly available databases. Reference genomes are used to determine the location of a sequence that is being analyzed in the organism's genome. As one having skill in the art would be aware, a consensus genome is a genome that is constructed from multiple genomes from the same species.

The term “sequence variation”, as used herein, is a variant that is different to an expected sequence or a reference sequence, such as a reference genome or sequence from a sample of a patient not anticipated to contain somatic variants, such as a buccal swab. A sequence variation may refer to a combination of a position and a type of sequence alteration. For example, a sequence variation can be referred to by the position of the variation and which type of substitution (e.g., G to A, G to T, G to C, A to G, etc. or insertion/deletion of a G, A, T or C, etc.) is present at the position. A sequence variation may be a substitution, deletion, insertion rearrangement of one or more nucleotides. In the context of the present method, a sequence variation can be generated by, e.g., a PCR error, an error in sequencing or a genetic variation. In many instances a sequence variation is a variation that is present at a frequency of less than 50% relative to other molecules in the sample. Many sequence variations, e.g., indels and nucleotide substitutions, are substantially identical to the molecules that do not contain the sequence variation. In some cases, a particular sequence variation may be present in a sample at a frequency of less than 20%, less than 10%, less than 5%, less than 1%, less than 0.5%, less than 0.1%, less than 0.05%, less than 0.01%, less than 0.001%, or less than 0.0001%.

The term “substantially” refers to sequences that are near-duplicates as measured by a similarity function, including but not limited to a Hamming distance, Levenshtein distance, Jaccard distance, cosine distance etc. (see, generally, Kemena et al, Bioinformatics 2009 25: 2455-65, the contents of which are hereby incorporated by reference in its entirety). The exact threshold depends on the error rate of the sample preparation and sequencing used to perform the analysis, with higher error rates requiring lower thresholds of similarity. In certain cases, substantially identical sequences have at least 98% or at least 99% sequence identity.

As used herein, the term “threshold” refers to a level of evidence (e.g., a ratio or set amount) that is required to make a call.

As used herein, the term “value” refers to a number, letter, word (e.g., “high”, “medium” or “low”) or descriptor (e.g., “+++” or “++”) that can indicate the strength of evidence. A value can contain one component (e.g., a single number) or more than one component, depending on how a value is analyzed.

Other definitions of terms may appear throughout the specification. It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

### DETAILED DESCRIPTION

The methods described herein result from the realization that cancer DNA can be reliably detected by incorporating and combining evidence from a variety of target regions containing one or more genetic variations, including single nucleotide variants, multiple nucleotide variants, copy number variants, short insertions and deletions, epigenetic variants, structural variants, and phased variants, into an assay of target regions. The methods have utility in enrichment-based methods examining target regions of the genome.

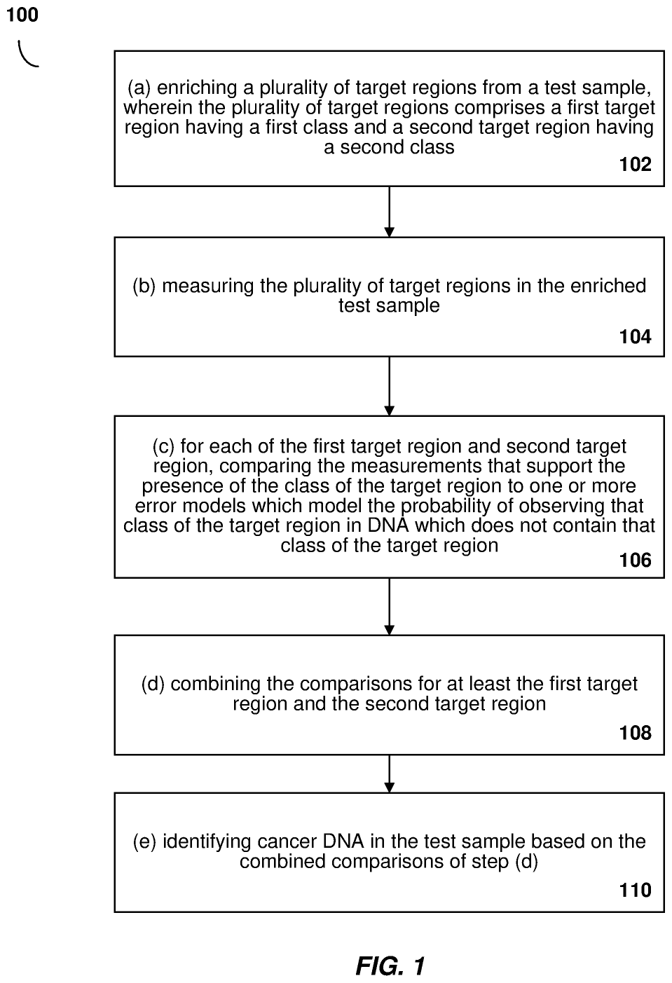

FIG. 1 depicts an embodiment of a method 100 of detecting cancer DNA in a test sample, such as a sample of blood collected from a cancer patient. In this embodiment, the method 100 can comprise (a) enriching a plurality of target regions from a test sample (step 102). The plurality of target regions can comprise a first target region comprising a first class and a second target region comprising a second class. The method 100 can continue by (b) measuring the plurality of target regions in the test sample (step 104). For each of the first target region and the second target region, the method 100 can continue by (c) comparing the measurements that support the class of the target region to an error model that models the probability of observing that class of the target region in DNA that does not contain that class of target region (step 106). The method 100 can continue by (d) combining the comparisons for at least the first target region and the second target region (step 108). The method 100 can continue by (e) identifying cancer DNA in the test sample based on the combined comparisons for the first target region and the second target region (step 110).

The methods disclosed herein may comprise a step of obtaining a test sample from a patient. Alternatively, the test sample may have been previously obtained from the patient. Test samples can comprise any nucleic acid sample or fluid containing DNA, RNA, or cDNA. Genomic DNA samples from a mammal (e.g., mouse or human) are types of test samples. Test samples may have more than about 10⁴, 10⁵, 10⁶ or 10^{7,} 10⁸, 10⁹ or 10¹⁰ different nucleic acid molecules. Any sample containing nucleic acid, e.g., genomic DNA or RNA from tissue culture cells or a sample of tissue, may be employed herein. Additionally, while many embodiments describe the detection or use of cancer DNA, methods according to the disclosure may be applied to any form of nucleic acid, and so the use of “cancer DNA” may equally refer to RNA or other detectable nucleic acids associated with cancer. Test samples can include blood plasma, blood serum, cerebrospinal fluid, urine, saliva, stool, amniotic fluid, aqueous humor, bile, breast milk, cerumen, chyle, exudates, gastric juice, lymph, mucus, pericardial fluid, peritoneal fluid, pleural fluid, pus, sebum, serous fluid, semen, sputum, synovial fluid, sweat, tears, vomit, or whole blood.

In some embodiments, the test sample comprises cell-free DNA (cfDNA), i.e. DNA that is free in a bodily fluid and not contained in cells. cfDNA can be obtained by centrifuging the test sample to remove all cells, and then isolating the DNA from the remaining liquid (e.g., plasma or serum). Such methods are well known (see, e.g., Lo et al, Am J Hum Genet 1998; 62:768-75). Circulating cell-free DNA can be double-stranded or single-stranded. The term cfDNA is intended to encompass free DNA molecules that are circulating in the bloodstream as well as DNA molecules that are present in extra-cellular vesicles (such as exosomes) that are circulating in the bloodstream. Cell-free DNA may contain cancer DNA, i.e., DNA that is from cancerous cells. Cancer DNA from a solid tumor can be found in cfDNA, in which case it may be referred to as tumor DNA ((DNA) or circulating tumor DNA (ctDNA). Cancer DNA can be identified because it contains mutations. In preferred embodiments, the test sample is cell-free DNA from the bloodstream (circulating cell-free DNA) which is DNA that is circulating in the peripheral blood of a patient.

In some embodiments, the test sample comprises cancer DNA isolated directly from a tissue biopsy, from circulating tumor cells (CTCs), or from other cells that are no longer part of the tumor tissue but are not circulating such as those in the urine or stool samples. In some embodiments, the test sample can comprise DNA isolated from cells, e.g., bone marrow cells, cells from a lymph node or circulating white blood cells, in the case of a blood cancer or cells from a lymph node, cells from a tumors margin or other sample types such as cerebrospinal fluid (CSF) and whole blood that are currently screened for the presence of cancer cells from solids tumors presently by other means. The cells may be obtained from a tissue sample (e.g., cancer tissue sample or suspected cancer tissue sample or tissue sample containing or suspected of containing a cancer cell) or fluid sample (e.g., any of the fluids listed above) from a patient.

The DNA molecules in cell-free DNA can be highly fragmented and may have a median size that is below 1 kb (e.g., in the range of 50 bp to 500 bp, 80 bp to 400 bp, or 100-1,000 bp), although fragments having a median size outside of this range may be present. Typically, cfDNA has a mean fragment size about 100-250 bp, e.g., 150 to 200 bp long, or about 160 bp. ctDNA is of tumor origin and originates directly from the tumor or from circulating tumor cells (CTCs), which are viable, intact tumor cells that shed from primary tumors and can enter the bloodstream or lymphatic system. The precise mechanism of how cancer DNA is released is unclear, although it is postulated to involve apoptosis and necrosis from dying cells, or active release from viable tumor cells. The amount of ctDNA in a sample of circulating cell-free DNA isolated from a cancer patient varies greatly: typical samples contain less than 10% ctDNA, although many samples from patients being assessed for MRD may have less than 0.01% ctDNA and some samples have over 10% ctDNA. Molecules of cancer DNA can be often identified because they contain tumorigenic mutations.

In one embodiment, the test sample is a blood plasma sample and cell-free DNA (cfDNA) is isolated from the blood plasma sample. The fraction of cancer DNA in the test sample (compared to non-cancerous DNA) may be equal or less than 0.01%, equal or less than 0.002%, equal or less than 0.005%, or equal or less than 0.001%. In some embodiments, a detectable fraction of cancer DNA in the test sample of DNA may be from about 0.0001%, however the actual limit of detection may vary. In some embodiments, the test sample comprises less than 25,000 genome equivalents of DNA (e.g., cfDNA), e.g., less than 20,000, less than 10,000, less than 5,000, or less than 1,000 genome equivalents of DNA. In some embodiments the test sample comprises from about 100 to about 25,000 genome equivalents (i.e., enrichable or amplifiable copies) of DNA. In some embodiments, the test sample comprises from about 10 ng to about 100 ng of DNA. In some embodiments, the test sample comprises at least 10 ng, at least 20 ng, at least 30 ng, at least 40 ng, at least 50 ng, at least 60 ng, at least 70 ng, at least 80 ng, at least 90 ng, or at least 100 ng of DNA. In some embodiments, the test sample comprises 66 ng of DNA.

Additionally, the methods described herein can be used to detect cancer DNA from both solid tumors and hematological (blood) cancers. Therefore, the term “cancer” can refer to any disease characterized by uncontrolled cell division and can be a blood cancer such as leukemia, lymphoma, or multiple myeloma, or a neoplastic cancer, e.g., associated with an abnormal mass of tissue in which cells grow and divide more than they should or do not die when they should. Neoplastic cancers, e.g., lung, breast, or liver cancer, are associated with a solid tumor. For solid tumor embodiments, the method may identify cancer DNA (here, tumor DNA) in cfDNA (e.g., circulating cfDNA). For blood cancer embodiments, the methods may identify cancer DNA in DNA extracted from cells taken from bone marrow, lymph node, or circulating white blood cells, or in cfDNA. For example, in blood cancer embodiments, one could take a bone marrow aspirate from an AML patient (pretreatment), determine the variants in their AML (e.g., by sequencing DNA from AML cells), and treat the patient. At some time after treatment, one could examine bone marrow aspirates, cell free DNA, or urine for evidence of those variants to determine whether the patient still has cancer. In some embodiments, the method may identify cancer DNA in tissue samples, such as surgical margins or lymph nodes.

A “target region” or “region” refers to a region of DNA that contains or is suspected of containing one or more genetic variations. Such a region, in reference to a genome or target polynucleotide, means a contiguous sub-region or segment of the genome or target polynucleotide. The term refers to any contiguous portion of genomic sequence whether it is within, or associated with, a gene, e.g., a coding sequence. A target region can be from a single nucleotide to a segment of a few hundred or a few thousand nucleotides in length or more. Typically, the length of a target region will be about or less than the average length of the nucleic acids present in a test sample. For example, in cfDNA embodiments, target regions will typically be about 160 bp. However, in other embodiments the length of a target region may be about 50 bp, 100 bp, about 200 bp, about 300 bp, about 400 bp, and about 500 bp. For example, in some embodiments in which the test sample is a tissue sample (e.g., from a lymph node or surgical margin), the length of a target region can be commensurate with the desired sequencing length or average fragment length. In practice, a target region can be any region targeted by a pair of PCR primers, and thus its length will be the length of the resulting amplicon.

Enriching a plurality of target regions (step 102) can be performed in a variety of ways, including but not limited to hybridization to nucleic acid probes, polymerase chain reaction (PCR), linked target capture, molecular inversion probes, ligation, and ATOM-Seq. In some embodiments, enrichment comprises capturing a plurality of target regions from the test sample by contacting the test sample with a pool of oligonucleotides. For example, the pool of oligonucleotides may contain oligonucleotides that comprise the reverse complement (or substantially the reverse complement) of the plurality of target regions. When the test sample is (e.g.) heated and the nucleic acids denature into single strands, the oligonucleotides may bind to any target regions and then be selected for (e.g., by a probe).

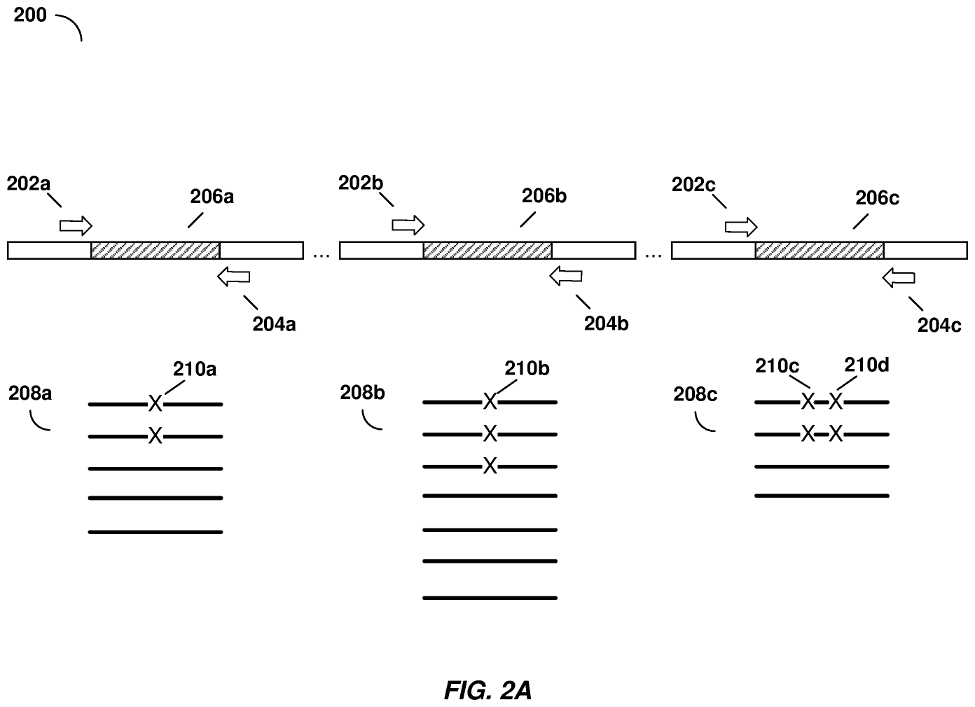

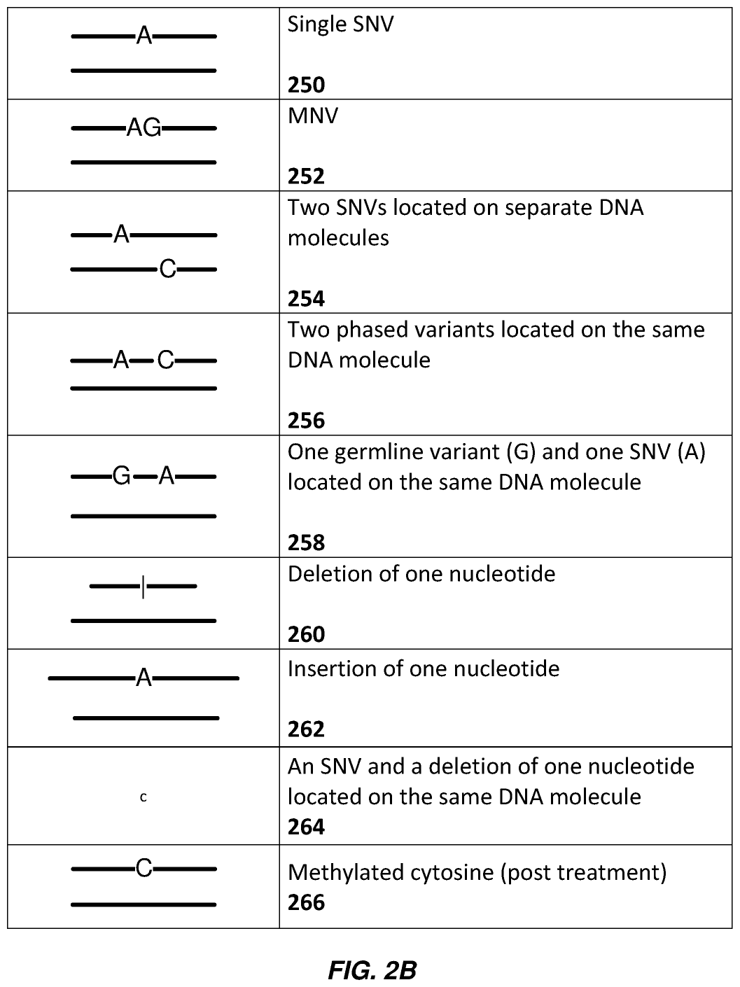

In some embodiments, enrichment comprises amplifying the plurality of target regions by polymerase chain reaction (PCR), i.e., an enzymatic reaction in which a specific template DNA is amplified using one or more pairs of sequence specific primers. As shown in FIG. 2, a forward primer 202a and reverse primer 204a can be designed to include sequences complementary to the beginning portion and end portion of a target region 206a. The forward and reverse primers are then added to a test sample including cancer DNA and subjected to PCR conditions including one or more rounds of thermocycling suitable for denaturation, renaturation, and extension with appropriate reagents (e.g., nucleotides, buffer, polymerase, etc.) as known in the art to produce a plurality of PCR products, such as amplicons 208a. The term “amplicon” as used herein refers to the product (or “band”) amplified by a particular pair of primers in a PCR reaction. The amplicons 208a may then be sequenced and the number of reads containing a sequence variation 210a may then be counted for that target region 206a.

The PCR may be a multiplex PCR employing two or more primer pairs for different targets. If the two or more targets are present in the reaction, a multiplex PCR results in two or more amplified DNA products that are co-amplified in a single reaction using a corresponding number of sequence-specific primer pairs. As shown in FIG. 2, a multiplex PCR can include three pairs of forward primers 202a, 202b, 202c and reverse primers 204a, 204, 204c which are individually designed for the plurality of target regions 206a, 206b, 206c, resulting in amplicons 208a, 208b, 208c which may then be sequenced. The observation of sequence reads containing sequence variations 210a, 210b, 210c provide support that the sequence variations observed are true genetic variations present in the sample, indicating the presence of cancer DNA in the test sample.

In some embodiments the test sample may first be pre-amplified, for example by whole genome amplification. Pre-amplification may be achieved, for example, by the ligation of adaptors and performing PCR targeting the ligated adaptors. In these embodiments, sequencing adapters may be added during amplification or may be ligated on after the amplification. In other embodiments, target regions may be enriched using a “target enrichment-based” approach in which adapters are ligated to the test sample, and fragments containing the target regions are enriched by hybridization to a nucleic acid probe prior to amplification using primers that hybridize to the adapters. In such embodiments, either ligation reactions may be performed, or adaptors with a plurality of barcodes may be ligated onto the DNA enabling the effective separation of groups of molecules into separate barcode groups or replicates. As such, sequences of the target regions can be enriched from the sample by PCR or by hybridization to a nucleic acid probe. Other enrichment methods may be used. In other embodiments any other method with either physical replication or use of molecular barcodes may be utilized such as Molecule Inversion Probes (MIP) or Anchored Multiplex PCR (AMP). In some embodiments the target regions may be enriched during the targeting step using methods including COLD-PCR, allele specific PCR, digestion of wild type sequence through the utilization of adjacent germline changes, or other methods known to those skilled in the art. In a preferred embodiment, the pre-amplification step is carried out with multiplex PCR, and the sample is then aliquoted into two or more samples for further PCR analysis (either singleplex or multiplex). In this embodiment, the sample may be pooled and subjected to a further barcoding step to enable sequencing of the amplicons.

While the remainder of the present disclosure describes in detail the use of PCR and “amplicon” sequencing, embodiments of the disclosure may also apply to other methods, including pre-amplified samples or methods that make use of molecular barcodes or indices, such as random sequences that are appended to a nucleic acid, pre-amplification. In such embodiments, comparing the measurements that support the presence of a class of target region to one or more error models can comprise estimating the probability of a sequence variation being present in a target region by (e.g.) measuring or counting the number of index sequences for that target region.

A “class” of target region can refer to a target region having one or more types of genetic variations. For example, a class of target region can comprise a target region containing: a single nucleotide variant (SNV), such as an A>T or C>G single base change; a multiple nucleotide variant (MNV), such as a CA>TG doublet base substitution or AAA>TTT triplet base substitution; a short insertion or deletion of one or more nucleotides (INDEL), such as an insertion of a TTTT or deletion of a CG; a copy number variant (CNV), including instances of gene amplification, chromosomal aneuploidy, or tandem repeats, which may often be detected as a target region having a significant increase in sequencing coverage; a structural variant (SV) reflecting a relatively large genetic change, such as gene fusions or large insertions or deletions of e.g., 1,000 s, 10,000 s, 100,000 s, or 1,000,000 s of nucleotides; and an epigenetic variant (EV), such as an alteration in DNA methylation, DNA-protein modifications, chromatin accessibility, histone modifications, and the like. In addition to the type of change, a class of target region can also refer to a specific change. For example, a class of target region can comprise an SNV change of A to T at a specific position or in a specific sequence context (such as a trinucleotide context, i.e. the specific nucleotides immediately surrounding a genetic variation or pentanucleotide context, i.e. the two adjoining bases either side of the change), an INDEL change of AAAA to AA, a SNV change of A to T at a first position and C to G at a second position, and the like.

A class of target region can also comprise multiple (i.e., two or more) genetic variations. The two or more genetic variations can be of the same type (e.g., two or more SNVs, INDELs, SVs, and EVs) or two or more different types (e.g., 1 SNV and 1 INDEL; 1 SNV and 1 INDEL and 1 EV; etc.). The two or more genetic variations may be separated by at least one nucleotide. Two or more genetic variations that are present on the same DNA molecule may be referred to as phased variants (PVs). The term “phased” refers to the determination of whether a genetic variation is positioned on either the maternal or paternal copy of that chromosome, e.g., chromosome 1. Two or more genetic variations may be considered PVs in the context of one another when they are both present on the same chromosome (i.e., the maternal or paternal copy), and thus would be present on the same DNA molecule in a test sample. If two PVs are sufficiently close together (e.g., within the same target region), they may be amplified and sequenced together and thus be observed on the same sequence read. As further illustrated in FIG. 2A, amplicons 208c generated from target region 206c can comprise two sequence variations 210c, 210d (each one individually denoted by an “X”) present on the same amplicon 208c, whereas amplicons 208a, 208b contain only single sequence variations 210a, 210b; thus, the two sequence variations 210c, 210d (if true genetic variations) are PVs. While a class of target region containing PVs may comprise any combination of phased genetic variations, in the context of cfDNA the class of target region containing PVs will often comprise two or more SNVs present on the same DNA molecule.

In some embodiments, the genetic variations are somatic variations, i.e., they are non-germline genetic variations that may be associated with a disease, such as cancer. In some embodiments, the genetic variations may include germline genetic variations, i.e., genetic variations that constitute the patient's (non-tumor) genome. Germline genetic variations may be useful in a target region class having two or more genetic variations. For example, a target region containing both a germline SNV and a somatic tumor SNV may be enriched and sequenced. Preferably, the germline SNV and tumor SNV are phased variants. In such cases, observing the tumor SNV, in combination with the germline SNV, within a single sequence read provides uniquely identifying information that increases the probability that the tumor SNV is real.

Various classes of target regions are further illustrated in FIG. 2B. For each example class in FIG. 2B, two DNA molecules are depicted as lines and illustrate the two copies for each chromosome (paternal and maternal) that would be amplified by a pair of PCR primers targeting a particular region. As shown in FIG. 2B, classes of target regions can comprise: an SNV (250); an MNV (252), here a doublet base substitution; two SNVs (254), positioned on opposite chromosomes and thus located on different DNA molecules; two PVs (256), which are two SNVs positioned on the same chromosome and thus located on the same DNA molecules; a germline SNV and a tumor SNV (258) which, as shown are PVs as they are positioned on the same chromosome and thus located on the same DNA molecules; a single INDEL, showing a deletion of a single base (260); a single INDEL, showing an insertion of a single base (262); an SNV and a deletion of a single base (264), which as shown are PVs as they are positioned on the same chromosome and thus located on the same DNA molecules; and an EV, showing a methylated cytosine which has not been converted to Uracil by way of bisulfite treatment (266).

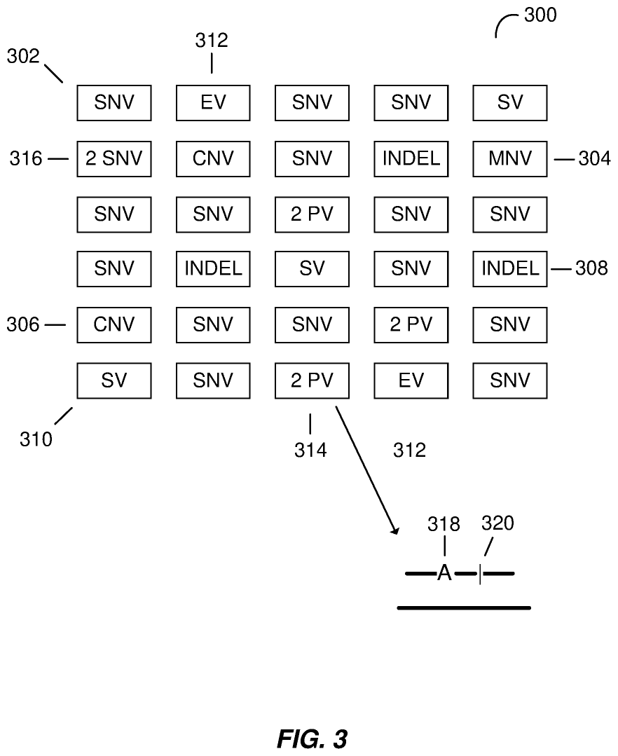

In some embodiments, enriching a plurality of target regions can comprise performing a multiplex PCR assay in which a plurality of target regions are simultaneously amplified in a test sample. FIG. 3 depicts an embodiment of a genetic variation profiling assay which uses a method as described herein 300. The assay 300 can comprise measuring a plurality of target regions, each target region comprising a class. As shown in FIG. 3, each box represents a different target region which is measured by the assay, preferably in the same reaction volume. The assay detects a number of different classes of target region, which can include: single nucleotide variations (SNVs) 302, multiple nucleotide variations (MNVs) 304, copy number variations (CNVs) 306, short insertions/deletions (INDELs) 308, structural variations (SVs) 310, epigenetic variations (EVs) 312, and phased variants (PVs) 314. As shown, some of the target regions can comprise two or more genetic variations, including 2 SNVs (316) (not on the same chromosome) and 2 PVs (314). Target regions having 2 SNVs on separate chromosomes have an advantage in that they double the utility of a particular target region by profiling two separate variations simultaneously, e.g., by using the same pair of PCR primers in a multiplex reaction. In some embodiments, either of the two SNVs or PVs may be a germline SNV. As previously noted, PVs can comprise any kind or combinations of genetic variation. For example, as further illustrated in FIG. 3, a 2 PV region (314) can comprise a region having both an SNV (318) and an INDEL (320) deletion of a single nucleotide.

Assays described herein can comprise any number of classes of target regions and may comprise multiple target regions having the same class. In preferred embodiments, an assay comprises at least two different classes of target regions. The assay 300 may be applied to a test sample to determine a status in the sample for each of the target regions profiled by the assay. In some embodiments, each target region may be enriched and measured (e.g., sequenced).). For any given target region, the measurements supporting the presence of the class of the target region (e.g., a specific SNV, CNV, INDEL, SV, EV, and/or two or more PVs located within a single target region) may be determined.

As will be described in further detail below, methods described herein can combine comparisons from multiple classes of target regions together, such as the multiple target regions containing genetic variations of the assay 300. One benefit of combining comparisons from various classes of target regions is that evidence from different variant types may be considered jointly to support a high-confidence conclusion that cancer DNA (or RNA) is present in a test sample. For example, in some embodiments a first target region comprises a first class, wherein the first class comprises a SNV. In these embodiments, a second target region can comprise a second class comprising a CNV, an INDEL, an SV, an EV, or, in particular, two or more PVs. In another embodiment, a first target region comprises a first class, wherein the first class comprises a CNV. In these embodiments, a second target region can comprise a second class, wherein the second class comprises a SNV, an INDEL, an SV, an EV, or two or more PVs, in particular two or more PVs. In some embodiments, a third target region can comprise a third class, wherein the third class comprises a SNV, a CNV, an INDEL, an SV, an EV, or two or more PVs, in particular two or more PVs. Various combinations of classes of target regions are contemplated herein.

Target regions may be selected by first identifying a plurality of genetic variations of interest, such as genetic variations associated with the patient's cancer. Genetic variations may include pre-identified sequence variations, such as variations known to be or suspected of being associated with the patient's cancer. The variations may also have been identified from the patient's cancer, such as somatic mutations that are in the genome of cells of the patient's cancer or were in the genome of cells of the patient's cancer prior to any cancer treatment. For example, genetic variations can comprise variations present or previously identified in various cancer-associated genes, including but not limited to TP53, EGFR, BRAF, and KRAS, and other genes frequently mutated in cancer (e.g., those in the COSMIC Cancer Gene Census, available at cancer.sanger.ac.uk/census; see also Sondka et al., The COSMIC Cancer Gene Census: describing genetic dysfunction across all human cancers, Nature Reviews Cancer 18, 696-705 (2018), the contents of which are hereby incorporated by reference); regions of common structural rearrangements (e.g., common gene fusions or the edges of common amplifications such as MYC), and regions of common amplification, rearrangements (e.g., Chromothripsis), common localized hypermutation (e.g., Kataegis), epigenetic changes, and the like.

In some embodiments, genetic variations can comprise cancer-specific genetic variations identified by sequencing DNA isolated from cancer cells from a patient. For example, cancer-specific variations can be identified by sequencing DNA or RNA isolated from a biological sample containing cancer cells obtained from a cancer patient. Tumor-specific variations can be identified by sequencing DNA or RNA isolated from a tissue sample obtained from a tumor biopsy from a cancer patient. Alternately, tumor-specific variants may be identified by sequencing cell-free DNA or RNA, or DNA or RNA isolated from circulating cancer cells from the patient. For blood cancers, genetic variations may be identified by sequencing a sample of DNA or RNA from bone marrow, circulating blood cells, or lymph nodes, for example. In such embodiments, an assay as described herein may be a “personalized” assay, in that the genetic variations are obtained from the same patient.

In some embodiments, cancer-specific variants are identified using targeted sequencing methods such as hybrid capture sequencing. In another embodiment, cancer-specific variants are identified using a pull-down or non-pull-down technique intended to enrich for selected sequences. These methods may sequence different areas of the genome such as the exome, i.e., whole exome sequencing (WES), which can include areas of the genome containing common mutations in cancer genes or areas containing frequent mutations that are not within genes. In preferred embodiments, cancer-specific variants are identified through WES of tumor tissue. In other embodiments, tumor-specific variants may be identified using whole genome sequencing (WGS) wherein a sample is sequenced without any specific enrichment.

WES and similar targeted sequencing methods effectively limit the search space across the genome by selecting for certain pre-identified sequences, yielding higher coverage and increased confidence in somatic variation calls. Such methods may also yield genetic variations more likely to have functional effects. However, limiting the search space may yield fewer total genetic variations, which can also influence the kinds of variations that are identified. For example, in blood cancers such as lymphoma, phased variants (PVs) tend to be clustered in known “hotspot” regions, and so can be identified using either targeted techniques (such as WES) or WGS. However, in solid cancers, PVs tend to be scattered randomly across the genome, and so fewer PVs that are sufficiently close to one another to be within a single target region will be identified. Accordingly, prior art methods focusing on identifying single types of variants, such as PVs, for use in a cancer diagnostic assay rely on WGS and identifying large numbers of PVs in “hotspot” regions in blood cancers (see, e.g., Kurtz, D. M. et al. Enhanced detection of minimal residual disease by targeted sequencing of phased variants in circulating tumor DNA, Nat Biotech 1-11 (2021), incorporated by reference herein in its entirety). The inventors have recognized and appreciated that PVs that are sufficiently close to one another can provide a significant increase in specificity, but only a few PVs within sufficient distance may be found using (e.g.) WES or targeted techniques in, e.g., methods of identifying genetic variations in target regions from solid tumors. Methods described herein solve this problem by combining measurements of target regions containing (e.g.) two or more PVs and target regions containing other kinds of variants, enabling a “hybrid” approach that investigates an economical number of genetic variations in a test sample and which can take advantage of any evidence available. For example, in the assay 300, evidence from a two PV target region 314 can be combined with evidence from a single SNV target region 302. In contrast, prior art WGS-based methods rely on identifying large numbers of PVs. Further, by focusing solely on phased variants, important information is missed; accordingly, a method that uses only phased variants as a class of target region would have less sensitivity than one that employs multiple classes.

In some embodiments, cancer-specific genetic variations are compared to genetic variations obtained from a matched normal sample. The sample is “normal” because it is derived from non-cancerous biological material and is “matched” because it is from the same patient. For example, a matched normal sample of non-cancerous DNA from the same patient may be sequenced, such as buccal swab DNA, whole blood DNA, or adjacent non-cancerous DNA (i.e., from tissue that is adjacent to a tumor that appears normal), and compared to the cancer-specific genetic variations of that patient. The sequencing of these matched normal samples may be performed at the same time as the sequencing of cancer cells from the patient or it may be performed before or after sequencing of cancer cells from the patient. Genetic variations that are detected in the cancer cells (cancer DNA) and not the matched normal samples (non-cancerous DNA) may be selected to be included in an assay as described herein, as these variations are more likely to be cancer-specific. Variations that are detected in the matched normal samples (non-cancerous DNA) may be excluded as they are likely to not be cancer-specific. One having skill in the art would be aware of various software packages for calling tumor-specific variants, such as MuTect2 (Cibulskis et al., Sensitive detection of somatic point mutations in impure and heterogeneous cancer samples. Nat Biotechnol. 2013; 31:213-9) and VarScan2 (Koboldt et al., et al. VarScan 2: somatic mutation and copy number alteration discovery in cancer by exome sequencing, Genome Res. 2012; 22:568-76).

In some embodiments, a genetic variation is a clonal genetic variation. Cancer DNA includes both clonal and sub-clonal mutations. In the evolution of a tumor, there is a transition between clonal and sub-clonal mutations. Sub-clonal mutations are only present in a subset of cells in the tumor: these occur after the most recent common ancestor of all cancer cells in the tumor sample. In contrast, clonal mutations occurred before the most recent common ancestor of all cancer cells. Clonal mutations are therefore present in all cells in the tumor unless there is some mechanism that has removed the mutation, e.g., a structural variation in which case the entire locus will be lost in a subset of cells. Clonal changes typically arise early in cancer evolution and are present throughout all of the cancers cells. A genetic variation may be considered clonal when it is present in multiple biological samples or can be inferred from sequence reads generated from bulk tumor tissue. Clonality can be difficult to determine as tumors are often heterogeneous, the entire tumor cannot be sequenced, and quantifying heterogeneity from bulk sequencing data is challenging. Various approaches have been proposed to determine clonality, including Bayesian mixture models, clustering probability distributions of cancer cell fractions, and phylogenetic methods. Software tools for determining clonality include PyClone-VI, EXPANDS, QuantumClone, and PhyloWGS. See also Gillis, S., Roth, A. PyClone-VI: scalable inference of clonal population structures using whole genome data. BMC Bioinformatics 21, 571 (2020); Andor et al., EXPANDS: expanding ploidy and allele frequencies on nested subpopulations, Bioinformatics 30(1): 50-60 (2013); Deveau et al., QuantumClone: clonal assessment of functional mutations in cancer based on a genotype-aware method for clonal reconstruction, Bioinformatics 34(11): 1808-1816 (2018); Deshwar et al., PhyloWGS: reconstructing subclonal composition and evolution from whole-genome sequencing of tumors, Genome Biology 16(35) (2015); the contents of each of which are incorporated by reference in their entireties. Samples may be sequenced by whole genome sequencing, whole exome sequencing, or targeted sequencing (e.g., by sequencing a panel of cancer genes or by sequencing a panel of sequences that are hotspots for mutations), etc.

The target regions containing the genetic variations may be ranked or filtered based on the types of genetic variations present. For example, target regions may be ranked based on one or more of: clonality, or allele fraction within a cancer sample; likelihood of a unique alignment; estimated background error rate, wherein genetic variations that show evidence of sequence or PCR polymerase error rate are penalized or filtered; high signal background events, wherein genetic variations that show DNA damage or early cycle PCR errors are penalized or filtered; the class of target region, such as prioritizing a pair of PVs given its predictive utility; proximity of any germline (not somatic) variants which may be helpful for enrichment; likelihood of being a somatic change; and the like.

Once a genetic variation has been selected, a corresponding target region may be determined by, e.g., selecting positions upstream and downstream of the genetic variation. For example, a target region can comprise a section of the genome that begins (e.g.) 75 bp prior to a genetic variation and ends (e.g.) 75 bp after the genetic variation. In some embodiments, PCR primers are designed to amplify a target region. In some embodiments, oligonucleotide probes are designed to enrich a target region. In embodiments where the test sample comprises cfDNA, preferably the target regions are designed to be about 150 bp in length, mirroring the average fragment length of cfDNA molecules.

Measuring the plurality of target regions (step 104) can be performed in a variety of ways. In some embodiments, measuring is performed by digital PCR (dPCR) or droplet digital PCR (ddPCR). Measuring may also be performed by quantitative PCR or other fluorescence-based assays. In some embodiments employing molecular barcoding, measuring can comprise generating a consensus sequence for a target region and determining whether the consensus supports the class of target region.

In some embodiments, measuring the plurality of target regions in the enriched sample comprises sequencing the plurality of target regions of step (a) to generate a plurality of sequence reads corresponding to the first target region and the second target region. In such embodiments, comparing the measurements (e.g., step 106 of method 100) comprises comparing the quantity of sequence reads that support the presence of the class of the target region to one or more error models that model the probability of observing that class of target region in DNA or RNA that does not contain that class of target region.

Sequencing generally refers to a method by which the identity of at least 10 consecutive nucleotides (e.g., the identity of at least 20, at least 50, at least 100 or at least 200 or more consecutive nucleotides) of a polynucleotide is obtained. In preferred embodiments, sequencing is performed using next-generation sequencing, i.e., the so-called highly parallelized methods of performing nucleic acid sequencing and comprises the sequencing-by-synthesis, sequencing-by-ligation, and sequencing by binding platforms currently employed by Illumina, Life Technologies, Pacific Biosciences, Element Biosciences, Singular Genomics, Omniome, Genapsys, Ultima Genomics, and Roche, etc. Next generation sequencing methods may also include, but not be limited to, nanopore sequencing methods such as offered by Oxford Nanopore or electronic detection-based methods such as the Ion Torrent technology commercialized by Life Technologies. In some embodiments, sequencing is performed using an Illumina NextSeq or NovaSeq system. In some embodiments, sequencing is performed using pyrosequencing, such as a Roche 454 GS FLX system.

The output of the sequencing process is a plurality of sequence reads, i.e., a string of letters indicating the order in which certain nucleotides (e.g., A, C, G, T) are present in a sequenced DNA molecule or amplicon. Sequence reads can vary in length from 25-1000 bp or more and, in many cases, each base of a sequence read may be associated with a score indicating the quality of the base call. As previously noted, cfDNA in blood is typically highly fragmented, with an average length of about 160 bp. Thus, in some embodiments, a target region may comprise about 160 bp in length and an amplicon may comprise about 160 bp in length or less. In these embodiments, the sequence reads are preferably at least 160 bp in length to sequence the entire amplicon and thus the entire target region.

In some embodiments, the sequence reads correspond to a first target region and a second target region. In some embodiments, sequencing adapters may be ligated directly on to amplicons having the sequence of the first target region and the second target region. In other embodiments, sequencing adaptors may be incorporated into the amplicons during amplification, i.e., during PCR. Various embodiments and modifications are contemplated within the scope of the disclosure.

In some embodiments, a target region comprises a class which comprises two or more phased variants (PVs). In such embodiments, two or more PVs are present (or expected to be present) within the same target region. As PVs are present on the same DNA molecule, a sequence read for the corresponding amplicon may include both PVs, providing highly specific evidence of cancer DNA being present in the sample.

In some embodiments, high depth of sequencing may be needed to identify genetic variations due to a low fraction of cancer DNA. In some embodiments, sequencing the plurality of target regions comprises sequencing to a minimum read depth of at least 10,000, at least 25,000, at least 50,000 or at least 100,000, at least 200,000 or at least 500,000. In some embodiments, sequencing the plurality of target regions comprises sequencing to a maximum read depth of at least 25,000, at least 50,000, at least 100,000, at least 200,000, at least 500,000, or at least 1,000,000. In any embodiment, the read depth of step may be from about 10,000 to about 500,000. In any embodiment, the read depth may be from about 10,000 to about 200,000.

In some embodiments, the sequence reads are processed computationally, e.g., by trimming, demultiplexing, aligning, matching, collapsing, and/or filtering. Typically, the processing will assign each of the sequence reads to one of the target regions that contains or is suspected of containing one or more genetic variations associated with the patient's cancer. For example, the sequence reads may be analyzed to identify which reads correspond to the plurality of target regions. As would be recognized by one of ordinary skill in the art, the sequence reads that are identical or near identical to the target region can be analyzed to determine if there is a potential genetic variation in the target sequence. Sequences may be aligned with a reference sequence, e.g., a genomic sequence, or matched to a database of expected sequences to determine their most likely location on the reference sequence.

After the sequence reads have been processed, the quantity (e.g., number) of sequence reads containing the genetic variation or plurality of genetic variations (k) and the total quantity (e.g., number) of sequence reads (n) may then be determined for each target region. Methods for quantifying reads may be adapted from those described by e.g., Forshew et al (Sci. Transl. Med. 2012 4:136ra68), Gale et al (PLoS One 2018 13:e0194630), and Weaver et al (Nat. Genet. 2014 46:837-843), all hereby incorporated by reference in their entirety. Similar results can be obtained using an approach that employs molecular indexes. In these methods the total number of molecules sequenced and the number of variant molecules can be estimated using the indexes. Such molecule identifier sequences may be used in conjunction with other features of the fragments (e.g., the end sequences of the fragments, which define the breakpoints) to distinguish between the fragments. Molecule identifier sequences are described in (Casbon Nucl. Acids Res. 2011, 22 e81), hereby incorporated by reference in its entirety.

Comparing the measurements that support the presence of the class of the target region to one or more error models that model the probability of observing that class of the target region in DNA that does not contain that class of the target region (step 106) can be performed in a variety of ways. In some embodiments, the comparing comprises comparing, for each target region, the measurements supporting the presence of a class (k) to a binomial, dispersed binomial, beta-binomial, multinomial, normal, exponential, or gamma error probability distribution model. For example, in one embodiment the error probability distribution model for a first class of target region is a beta-binomial error probability distribution model and the error probability distribution model for a second class of target region is a multinomial error probability distribution model. In some embodiments, the number of sequence reads supporting the presence of a class (k) comprises the number of sequence reads containing a genetic variation. In some embodiments, the comparing further comprises generating a statistical assessment or score describing the degree of evidence supporting a conclusion that a given target region contains the one or more genetic variations in the sample. In some embodiments, the statistical assessment can be, e.g., a p-value, likelihood, likelihood ratio, or a probability distribution. A statistical assessment may also preferably include a likelihood ratio approach in which the likelihood of observing n sequence reads containing the one or more genetic variations in the test sample is determined if i) there is cancer DNA in the sample, and ii) there is not cancer DNA in the sample. These values may then be used to calculate (e.g.) a likelihood ratio to determine whether the one or more genetic variations in a target region are present in the sample.

As previously noted, cancer DNA, if present, will often represent a small fraction of cell-free DNA. For example, in MRD, the cancer fraction may be as low as 0.01 ppm. At this level, the inventors have recognized and appreciated several issues which can create, for example, false positive results. First, sequencing is not perfect, and background error may result in a misread base potentially leading to a false positive ctDNA call. Second, errors may also be introduced during PCR. For example, a base may be “switched” due to DNA damage (e.g., oxidation, deamination) prior to amplification, and the subsequent amplification by PCR may eventually result in many sequence reads supporting an incorrect conclusion. Additionally, as the number of genetic variants included in an assay increases, the possibility of a false positive call rises accordingly. Some assays require (e.g.) calling at least two individual genetic variations as positive for an accurate diagnosis. However, this approach can be flawed in that it limits the number of variants tested by an assay, thus reducing the amount of available ctDNA “signal” and decreasing sensitivity.

One way to account for background error is to model the error as a probability distribution and then determine whether an observed genetic variation is unlikely to come from the background error. For example, the probability of observing k sequence reads containing a genetic variation in a target region given a background error rate p can be determined using a binomial probability distribution:

$$P ( X = k ) = Binom ( k , n , p ) = ( \frac{n}{k} ) {{p}^{k} ( 1 - p )}^{n - k} , \qquad ( 1 )$$

where n is the total number of sequence reads. The background error rate may be estimated, e.g. from a set of control samples not containing any cancer-associated variants. One may call the genetic variation as present in the sample if the determined probability is less than a threshold level (e.g., 0.05, 0.01, 0.001, 0.0001).

A probability (e.g., P (X=k) refers to the chance of a particular outcome occurring, or how likely that outcome is to occur. Probability may be based on the values of parameters in a model. Probability refers to unknown events and attaches to possible results. Since possible results are mutually exclusive and exhaustive, a probability can be expressed on a linear scale. For example, a probability may be expressed as a value between 0 (impossible) and 1 (certain) or may equally be expressed as a percentage or fraction. For example, in the context of the present disclosure, a probability may be used as a measure to determine whether cancer DNA is present in a sample

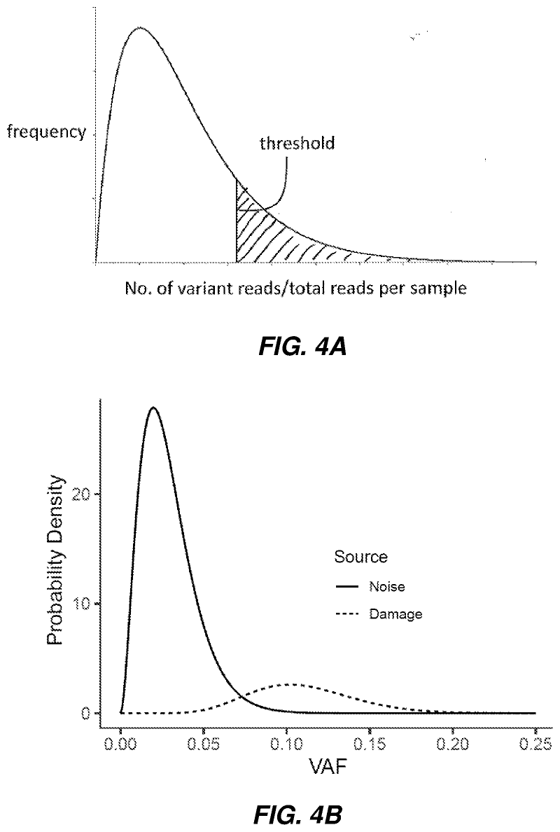

An error probability distribution (which may also be called an “error model”, “error distribution”, or “error probability distribution model”) refers to a distribution that estimates or models the probability that an observation (such as a variant allele fraction) is due to error. These terms can refer to any kind of error, including error attributed to DNA damage or early cycle PCR errors, as well as sequencing errors. Hypothetical error models are shown as frequency distributions in FIGS. 4A-B. In these examples, multiple samples (e.g., several hundred samples) that are not known to contain somatic genetic variations (i.e., healthy control samples) are sequenced, and the fraction of sequence reads that have a particular type of sequence variation is calculated for each sample. Any sequence variations within the sequence reads are largely caused by errors that occur during PCR, base miscalls, and pre-PCR events such as DNA damage (e.g., the oxidation of guanine to 8-oxoguanine, which base pairs with A, resulting in what appears to be a G to T variation in a sequence read). These fractions can be plotted as a frequency distribution which, in turn, can be used to calculate the probability of whether a sequence variation observed in a sequence read is really a genetic variation.

In some embodiments, a likelihood ratio (LR) may be used to estimate the degree of evidence supporting the presence of a class of a target region. A likelihood ratio refers to a ratio of at least two likelihoods, each attached to a different hypothesis, which can be used to determine which hypothesis is more likely given an experimental result. Each likelihood refers to the hypothetical probability of a specific outcome being yielded by an event that has already occurred. Likelihood is used to assess how well a sample provides support for particular values of a parameter in a model. Likelihood therefore refers to past events with known outcomes and attaches to hypotheses.

Likelihood ratios can be used as a measure of diagnostic accuracy since they can be used to determine the potential utility of a particular diagnostic test, and how likely it is that a patient has a disease or condition. As applied to a diagnostic test, a likelihood ratio is the likelihood that a given result would be expected in a sample not having any cancer DNA compared to the likelihood that the same result would be expected in a sample containing cancer DNA. As shown in Equations (2), (3), and (4) below, two hypotheses may be determined: H₀, the likelihood of observing k reads containing a genetic variation assuming that there is no cancer in the sample (the null hypothesis), and H₁, the likelihood of observing k reads containing the genetic variation (and thus supporting a class of a target region) assuming that there is at least one cancer molecule present (where z is the total number of input DNA molecules, which may be estimated, e.g. by optical diffraction or digital PCR). Each hypothesis incorporates a background error rate (p) indicating the frequency at which a sequence read containing the genetic variation in that class of target region may be due to error. The background error rate (p) may individually selected for each target region. The ratio of these two values (H₁/H₀) may then be determined and compared to a threshold. A value more than 1 suggests that H₁ is the more likely hypothesis, whereas a value between 0 and 1 suggests that H₀ is more likely. One can vary the threshold required to call a cancer-associated variant as present depending on the desired sensitivity and specificity, which may be determined from (e.g.) a set of known samples.

$${H}_{0} = Binom ( k , n , p ) \qquad ( 2 )$$

$${H}_{1} = Binom ( k , n , p + \frac{1}{z} ) \qquad ( 3 )$$

$$LR = \frac{{H}_{1}}{{H}_{0}} \qquad ( 4 )$$

Accordingly, in any embodiment, comparing the number of sequence reads that support the presence of a class of a target region (k) to one or more error models (step 106) can comprise calculating a likelihood ratio between the likelihood of observing the number of sequence reads containing a genetic variation: (i) if cancer DNA is present, and (ii) if cancer DNA is not present. Along similar lines, in any embodiment this may be done by calculating a likelihood ratio (LRᵢ) between the likelihood of observing k reads for each target region: (i) if cancer DNA is present and (ii) if cancer DNA is not present. As will be described in more detail below, in these embodiments, the individual likelihood ratios LRᵢ may be combined into a cumulative LR score (e.g., the product of LRᵢ equivalent to the sum of log-likelihoods) across all target regions of a test sample.

In some embodiments, a target region class comprises two or more phased variants (PVs). The two or more PVs can comprise a first genetic variation and a second genetic variation positioned on the same DNA molecule. Each variation would thus be sequenced together on a single sequence read. In these embodiments, comparing the quantity of sequence reads that support the presence of a class of a target region, wherein the class comprises two or more phased variants, to one or more error models that model of observing that class of target region in DNA that does not contain that class of target region (step 106) can be performed by comparing the number of sequence reads containing both the first genetic variation and the second genetic variation, the number of reads containing only the first genetic variation, the number of reads containing only the second genetic variation, and the number of reads containing neither genetic variation to a multinomial distribution. For example, the probability of observing two phased variants in a target region can be modelled as:

$$P ( {k}_{1} , {k}_{2} , {k}_{3} , {k}_{4} ) = \frac{( {k}_{1} + {k}_{2} + {k}_{3} + {k}_{4} ) !}{{k}_{1} ! {k}_{2} ! {k}_{3} ! {k}_{4} !} {X}_{1}^{{k}_{1}} {X}_{2}^{{k}_{2}} {X}_{3}^{{k}_{3}} {X}_{4}^{{k}_{4}} , \qquad ( 5 )$$

where k₁ is the number of sequence reads where both genetic variants are observed (Alt, Alt), k₂ is the number of sequence reads where only the first genetic variant is observed (Alt, Ref), k₃ is the number of reads where only the second genetic variant is observed (Ref, Alt), k₄ is the number of reads where neither variant is observed (Ref, Ref), and X=(X₁, X₂, X₃, X₄) is a random vector whose components are not independent, but satisfy a condition in that they sum to one.

Various probability density functions can be used for X. One candidate for this role is the standard Dirichlet distribution:

$$f ( {x}_{1} , {x}_{2} , {x}_{3} , {x}_{4} ) ∝ δ ( {x}_{1} + {x}_{2} + {x}_{3} + {x}_{4} - 1 ) ( {x}_{1} + {x}_{2} + {x}_{3} + {x}_{4} - 1 ) \times \frac{Γ ( {α}_{1} + {α}_{2} + {α}_{3} + {α}_{4} )}{Γ ( {α}_{1} ) Γ ( {α}_{2} ) Γ ( {α}_{3} ) Γ ( {α}_{4} )} {x}_{1}^{{α}_{1} - 1} {x}_{2}^{{α}_{2} - 1} {x}_{3}^{{α}_{3} - 1} {x}_{4}^{{α}_{4} - 1} \qquad ( 6 )$$

where δ(−) is the Dirac delta distribution, α₁, α₂, α₃, α₄≥0 are parameters, and 0≤x₁, x₂, x₃, x₄≤1. This distribution has an important property in that the marginal distributions are beta distributions:

$${X}_{i} ∼ Beta ( {α}_{i} , {α}_{0} - {α}_{i} ) \qquad ( 7 )$$

where α₀=α₁+α₂+α₃+α₄, for i=1, 2, 3, 4. One may also consider using a more advanced model (such as the Generalized Dirichlet distribution) to capture more complicated correlations between the components of X.

Individual probabilities may also be calculated for X. For example, Xᵢ=P(kᵢ), i.e. the probability of observing kᵢ reads. Given a tumor fraction θ, the probability of observing individual kᵢ's are:

$$Alt , Alt : P ( {k}_{1} ) = ( 1 - θ * {e}_{1} * {e}_{2} ) + ( θ * 1 - {e}_{1}^{′} * 1 - {e}_{2}^{′} ) \qquad ( 8 )$$

$$Alt , Ref : P ( {k}_{2} ) = ( 1 - θ * {e}_{1} * 1 - {e}_{2} ) + ( θ * 1 - {e}_{1}^{′} * {e}_{2}^{′} ) \qquad ( 9 )$$

$$Ref , Alt : P ( {k}_{3} ) = ( 1 - θ * 1 - {e}_{1} * {e}_{2} ) + ( θ * {e}_{1}^{′} * 1 - {e}_{2}^{′} ) \qquad ( 10 )$$

$$Ref , Ref : P ( {k}_{4} ) = ( 1 - θ * 1 - {e}_{1} * 1 - {e}_{2} ) + ( θ * {e}_{1}^{′} * {e}_{2}^{′} ) \qquad ( 11 )$$

Where e₁ and e₂ are error rates for a first and second phased variant (respectively) where an observation of the first and second PV comes from non-cancerous DNA and is due to sequencing error, and e₁′ and e₂′ are error rates where a corresponding observation of non-cancerous DNA at the positions of the first and second phased variant comes from tumor DNA and is attributed to sequencing error. In such embodiments, the error rates may be replaced with random variables (e.g. a binomial or beta-binomial distribution), yielding a random variable P(θ):

$$P ( θ ) = mean ( P ( {k}_{1} , {k}_{2} , {k}_{3} , {k}_{4} ) ) , \qquad ( 12 )$$

which may be incorporated into a hypothesis of cancer DNA being present in the sample (H₁) as described herein.

While the above embodiment describes two phased variants, methods according to the disclosure may be further modified to account by one of skill in the art to accommodate target regions having additional PVs, such as three or more, four or more, five or more, or ten or more PVs. However, in preferred embodiments two PVs is typically sufficient for a target region class, as these are more likely to be discovered in close proximity to one another and present on a single DNA fragment.

Various error models may be used to model the probability of observing a class of target region in DNA that does not contain that class of target region. In some embodiments, an error model is selected corresponding to a class of target region. In some embodiments, the class of target region is an SNV and the error model is a binomial distribution. In some embodiments, the class of target region is two or more PVs and the error model is a multinomial distribution. In some embodiments, the comparing comprises comparing to two or more error models. In such embodiments, the two or more error models can model various types of error including but not limited to sequencing error, PCR error, DNA damage, polymerase error, and the like. For example, a first error model (e.g., a binomial probability distribution) can be used to estimate a background error rate from sequencing and a second error model (e.g., a Poisson distribution) can be used to estimate a background error rate from DNA damage. In some embodiments, a single distribution may account for one or more types of error. For example, the two shape parameters (α, β) in a beta-binomial distribution may be tuned to accommodate an estimated background error rate and DNA damage.

In some embodiments, the background error rate is estimated using a probability distribution. In some embodiments, there may be two distributions of the same family or type (e.g., 2 binomial distributions) or, if two different families or types of distribution are used, there may be one distribution for the background error rate and another for PCR errors.

In some embodiments, H₁, the likelihood of observing k reads containing the genetic variation (and thus supporting a class of a target region) assuming that there is at least one cancer molecule present, is adjusted by an additional probability, such as the probability that a genetic variation is cancer-specific (V_{C}). For example, equation (3) above can be modified as follows:

$${H}_{1} = P ( {V}_{c} ) * Binom ( k , n , p + \frac{1}{z} ) \qquad ( 13 )$$

This approach is useful in embodiments incorporating (e.g.) structural variants, INDELs, and phased variants as it is highly unlikely that any background error is responsible for observing such genetic variations. In such cases, a single sequence read may provide a significant amount of evidence that there is cancer DNA present in the test sample. Accordingly, in such embodiments it may be desirable to modify H₁ by the probability that the genetic variation may not be tumor specific (V_{C}), which may be due to clonal hematopoietic mutations of indeterminate potential (CHIP) or potential contamination. In preferred embodiments, V_{C} is set to a value such that only a single observed sequence read supporting the presence of a class is insufficient to provide a large amount of evidence supporting a conclusion of cancer DNA in the sample.

Additionally, the inventors have recognized and appreciated that short insertions and deletions (INDELs) may be particularly useful when included as a class of target region according to an embodiment of the disclosure. For example, a target region class can comprise an INDEL of, e.g., 1-5, 1-10, 1-15, or 1-20 nucleotides in length. In such embodiments, INDELs of 1-5 nucleotides are preferred as these are more likely to be observed in a test sample. In other embodiments, a target region class can comprise INDELs of 1 or more, 2 or more, 3 or more, 4 or more, 5 or more, 6 or more, 7 or more, 8 or more, 9 or more, 10 or more, or 20 or more nucleotides in length. In some embodiments, a maximum INDEL length is 20 nucleotides as longer changes may affect the accuracy of sequence alignment. Depending on the type of sequencing technology used, the background error rate associated with INDELs may be very low. Accordingly, observing an INDEL in a target region may provide a relatively large amount of evidence that the INDEL (and thus, cancer DNA) is present in the test sample.

Error models may be trained using control samples, such as DNA samples which are known to not contain any sequence variations or which have been collected from healthy patients (e.g., patients not having cancer). By sequencing control samples known to not contain any sequence variations, any observed sequence variations in the control samples must be due to error. Such observations may be used to set the parameters of an error model according to the disclosure. Preferably, control samples are processed under similar conditions as a test sample. For example, the primers may amplify the same or similar target regions and the sequencing technology may be the same. Many control samples may be used to build an error model, such as at least about 50 samples. Error models can be stored in a computer database and accessed as needed. Thus, in one embodiment, an error model is trained based on a set of control samples. In one embodiment, the set of control samples are from healthy donors. In such embodiments, training an error model based on a set of control samples establishes the background error (p) for the class of target region in the absence of cancer.

In some embodiments, a multiple-comparison correction is applied to a comparison to prevent false positives, such as setting a more stringent threshold or applying a Bonferroni correction. In any embodiment, the threshold may be determined using a binomial, over-dispersed binomial, Beta, Normal, Exponential or Gamma probability distribution model of the background error rate for the sequence variation and wherein the frequency is selected such that a signal would be observed above this less than 0.1%, 0.01% or 0.001% of the time, preferably 0.1% of the time, depending on the desired pre-defined per variant specificity, when no mutant molecules are present.

In some embodiments, CNVs are identified using a read depth approach in which a non-overlapping sliding window is used to count the number of sequence reads that are mapped to a genomic region overlapping the window. Regions with a significant increase in read depth (more than expected according to typical background error associated with sequences) may be further analyzed to identify copy number. Alternately, a paired-end approach may be used in which copy number variations are detected based on distances between mapped paired sequence reads. Sequence reads may also be assembled de novo and the resulting assembled contiguous sequences may be aligned to the reference genome to identify copy number variation.

In some embodiments, epigenetic variants (EVs) are identified by treating a test sample and then sequencing. For example, methylated nucleotides can be identified by treatment with sodium bisulfite, which converts unmethylated cytosine to uracil. Amplification and sequencing of the sample then converts the uracil bases to thymine (T). Thus, the presence of an unmodified cytosine base in a target region can support the presence of a target region containing an EV. The comparing for EVs may be performed using error models that similarly measure the background level of sequencing error, optionally accounting for any error associated from the bisulfite conversion process.

Combining the comparisons for at least the first target region and the second target region (step 108) can be performed in a variety of ways. In some prior art methods, each genetic variation is called individually rather than for a set as a whole. While statistical corrections may be applied on individual variant calls as the number of variants increases, the higher stringency required to eliminate false positives may also have an adverse effect on sensitivity and discount most variant calls. The inventors have recognized and appreciated that each independent analysis of each target region can contribute some level of evidence to a cumulative statistical assessment. Rather than considering each target region individually, combining scores from two or more target regions can yield high confidence calls for a test sample without a corresponding decrease in sensitivity or an increase in false positives. Accordingly, in some embodiments, the comparing for each of the first target region and the second target region (and any other target regions considered) may be accumulated into a score or statistical assessment measuring the overall degree of evidence supporting a conclusion of cancer DNA being present in the test sample. In some embodiments, the comparisons are combined to yield a cumulative statistical assessment representing the probability or likelihood of cancer DNA being present in the test sample. Various methods may be used to create a cumulative statistical assessment, including a joint statistical measure (such as a joint probability, joint likelihood, or joint likelihood ratio) or otherwise combining (e.g., summing, averaging) the result for each target region to identify whether cancer DNA is present in the test sample.

In some embodiments, the combining comprises calculating an average of each of the comparisons. In one embodiment, the average is a weighted average. For example, a comparison from a first target region having a class of two or more PVs may be given a weight of 1.0, whereas a comparison from a second target region having a class of a single SNV may be given a weight of 0.5. In this way, the two or more PV class provides additional weight as the probability of observing two genetic variations together on a single sequence read is less likely to be the result of error. Accordingly, in one embodiment, combining the comparisons for at least the first target region and the second target region (step 108) can further comprise adjusting each of the comparisons by a weight. In some embodiments, the weight for a class of two or more PVs is 1.0. In some embodiments, the weight for a class of a single SNV is 0.5. In some embodiments, each of the comparisons may have been performed using different calculations, such as by using different error models or statistical techniques to evaluate each target region. For example, in some embodiments, a first target region having a class of a single SNV uses an error model derived from a binomial distribution and a second target region having a class of two or more PVs uses an error model derived from a multinomial distribution. In such embodiments, different weights may be applied to each type of comparison such that the evidence supporting a call of cancer being present is proportional to the statistical assessment being made.

In some embodiments, the comparisons comprise generating a statistical assessment, such as a p-value, describing the probability or likelihood of a genetic variant being present in the test sample. In these embodiments, p-values for each target region may be combined using, e.g., Fisher's method. If U is distributed as Uniform(0,1), then −2logU is distributed as Chi-square with 2 degrees of freedom (X₂²). If X₁, . . . , Xₖ are independently distributed as Xᵥ_{₁}², . . . , Xᵥ_{ₖ}², then X₁+ . . . +Xₖ is distributed as Xᵥ_{₁}_{+ . . . +v}_{ₖ}². Since p₁, . . . , pₖ are independently distributed as Uniform(0,1), then the combined p-value is:

$$p = P ( {X}_{2 k}^{2} \geq - 2 {\sum }_{i = 1}^{k} log {p}_{i} ) \qquad ( 14 )$$

By way of example, if there are three target regions having independent statistical assessments (e.g., p-values) of 0.145, 0.263, and 0.081, a combined statistical assessment can be:

$$p = P ( {X}_{2 ( 3 )}^{2} \geq - 2 [ log ( 0.145 ) + log ( 0.263 ) + log ( 0.087 ] ) = P ( {X}_{6}^{2} \geq 11.417 ) = 0.0763 ,$$

which, as shown, provides moderate evidence against the null hypothesis. Additional methods for combining statistical assessments from multiple independent statistical tests can be found at least in Jiang and Wong, Open Journal of Statistics Vol. 5 No. 01 (2015), hereby incorporated by reference in its entirety.

In some embodiments, the statistical assessment can comprise a likelihood ratio indicating the likelihood of a genetic variant being present in a test sample. Individual likelihood ratios may be combined into a cumulative LR score (product of LRᵢ equivalent to sum of log-likelihoods) across all target regions of a sample. In these embodiments, the likelihoods or likelihood ratios can be combined by finding the sum of the log-likelihoods of each individual event, i.e., the likelihood calculated for each target region. In this way, when parameters are estimated using the log-likelihood for the maximum likelihood estimation, each data point is used by being added to the total log-likelihood. Thus, each data point is evidence that supports the estimated parameters, in which the addition of each data point adds independent evidence to identify whether there is cancer DNA present in the sample.

In one embodiment, log likelihoods or log likelihood ratios may be determined for each genetic variation and then combined, e.g. by summing. As shown in Equation (6) below, the likelihood ratio for the entire sample may be determined by summing the log likelihood ratios for each of the target regions (1 . . . V) being considered:

$${LR}_{Sample} = {\sum }_{i = 1}^{V} log {LR}_{i} \qquad ( 15 )$$

Similarly, one may sum the log likelihoods for each of the H₀ and H₁ determinations for each variant and then find the likelihood ratio:

$${LR}_{Sample} = \frac{{\sum }_{i = 1}^{V} log {H}_{{1}_{i}}}{{\sum }_{i = 1}^{V} log {H}_{{0}_{i}}} \qquad ( 16 )$$

In this way, scores for each of the individual target regions are not simply compared to a threshold individually. Rather, the scores from every target region in the assay are considered jointly. This provides a further benefit in that evidence from different classes of target regions can also be considered jointly.

By considering evidence from multiple target regions jointly, embodiments described herein can scale to any number of target regions that is practical. For example, while the assay 300 of FIG. 3 comprises 30 target regions, embodiments of the disclosure can scale to 1-100 target regions, 100-200 target regions, 200-300 target regions, 300-400 target regions, or 400-500 target regions. In some embodiments, the number of target regions is 500-1000 target regions, 1000-2000 target regions, 2000-3000 target regions, 3000-4000 target regions, or 4000-5000 target regions. In some embodiments, the number of target regions is 5000-10,000 target regions, 10,000-20,000 target regions, 20,000-30,000 target regions, 30,000-40,000 target regions, or 40,000-50,000 target regions. In some embodiments, the number of target regions is proportional to the cancer type. For example, melanomas may have up to a million SNVs, whereas most cancers have about 100,000. Accordingly, in these embodiments, the number of target regions can comprise 50,000-100,000 target regions, 100,000-200,000 target regions, 200,000-300,000 target regions, 300,000-400,000 target regions, 400,000-500,000 target regions, or 500,000-1,000,000 target regions. In any embodiment the number of target regions is at least 2, at least 4, at least 10, at least 20, at least 50, at least 100, at least 500, at least 1000, or at least 5,000 target regions. In many embodiments, 2-200, e.g., 6-100, target regions may be examined.

Identifying cancer DNA in the test sample based on the combined comparisons for the first target region and the second target region (step 110) can be performed in a variety of ways. In some embodiments, identifying cancer DNA in the test sample can comprise comparing a cumulative assessment of a plurality of target regions to a threshold value. In any embodiment, cancer DNA may be identified or otherwise considered present in the test sample based on the cumulative assessment. For example, cancer DNA may be identified in the test sample if the cumulative assessment exceeds a threshold value. Appropriate threshold values may be determined empirically, e.g., by sequencing a quantity of test samples in which it is previously known whether there is cancer DNA present, and then selecting a threshold value that has both a high sensitivity (i.e., ability to detect) and high specificity (i.e., ability to discriminate). In such embodiments, the threshold may be determined by running at least 10, at least 100, at least 1000, or at least 10,000 samples comprising non-cancerous DNA (or at least are not known to have cancer DNA) through the assay and selecting a threshold above the signal identified in the control samples or a threshold such that the false positive rate as determined using the control samples is estimated to be 1% or below, 0.1% or below, or 0.01% or below. The samples which are run may be from the same patient or they may be from different patients. For example, running 200 samples may involve taking a sample from 20 healthy donors (assumed to not have cancer) and running 10 assays per patient to reach 200 samples. For each control sample the likelihood ratio analysis may be applied to give an overall likelihood ratio for a healthy patient. Calculating the likelihood ratio for all the samples which have been run results in a range of likelihood ratios for a healthy patient and the threshold can be set somewhere above the highest likelihood ratio. This threshold may be calculated from a pool of healthy donors in advance and therefore does not change on a patient-by-patient basis. As would be apparent, methods according to the disclosure may further comprise identifying the patient as having cancer if the result is at or above the threshold and, for example, administering a therapy to the patient. In these embodiments, the patient may have previously undergone a first therapy. In these cases, the method comprises administering to the patient a second therapy that is different to the first therapy.

As previously noted, the inventors have recognized and appreciated that different classes of target regions including different kinds of variants can provide various levels of evidence to support an identification of cancer DNA in a test sample. For example, observations of phased variants and INDELs (particularly INDELs longer than 1 nucleotide, e.g., 2, 3, 4, 5 nucleotides or more, preferably 2 nucleotides or 3 nucleotides) within a target region are highly unlikely to be caused by background error. In contrast, single nucleotide variations may contribute only a relatively low amount of evidence towards a conclusion of cancer DNA being present as these types of variations are more likely to be the result of error. Accordingly, assays according to the disclosure may combine results from multiple classes of target regions together to take advantage of all tumor-associated variants that may be available, resulting in a highly sensitive and highly specific cancer DNA detection assay.

In some embodiments, a test sample may be divided into two or more aliquots, which may be processed according to embodiments of the disclosure in the same manner. For example, each aliquot may be similarly enriched, measured, and compared for certain target regions, which comparisons may be combined to yield a high confidence identification of cancer DNA present in the test sample. More information on aliquoting and the use of replicate samples may be found in commonly owned International Patent Application No. PCT/IB2022/051195, filed on Feb. 10, 2022, which is hereby incorporated by reference in its entirety.

In any embodiment, a variant allele fraction (VAF) may be determined for the test sample. The VAF may be determined, e.g., by the quantity of sequence reads supporting the presence of a class of a target region. The term “variant allele fraction”, “estimated variant allele fraction”, “VAF, or “eVAF” refers to the estimated allele fraction of variant cancer DNA within a test sample.

In any embodiment, the amount of cancer DNA within the test sample may be quantified. Quantification may include an estimated variant allele fraction. In some embodiments, the estimated allele fraction can comprise a mean or median of the variant allele fraction for each target region in which it was determined that the class of target region was present. In some embodiments, the estimated variant allele fraction can comprise a mean of the variant allele fraction (k/n) for each variant. This can be preferable in situations where variant levels are low and the results are stochastic, and therefore including evidence from all variants may result in a more realistic measure. Quantified cancer DNA may be compared to the quantified cancer DNA from one or more additional samples, such as comparing quantified cancer DNA from samples obtained from a patient during at least a first time point and a second time point, wherein the first time point is prior to a treatment and the second time point is after a treatment. Similarly, one could track individual variants or groups of variants across samples from different time points.

In any embodiment, methods described herein may be performed on test samples that are obtained from the patient during at least a first time point and a second time point, wherein the first time point is prior to a therapy and the second time point is after the therapy, and the method comprises determining if there is a change in the amount of cancer DNA or a range of likely amounts of cancer DNA between the first and second time points. In any embodiment, further samples may be obtained at additional time points, for example wherein additional samples are taken after the second time point on a monthly, bimonthly, quarterly, or annual schedule. This embodiment may be used to monitor whether a therapy being administered to the patient is continuing to be effective. The change in cancer DNA over time may be determined using point estimates, confidence intervals or both, and wherein a significant (e.g. a statistically significant) decrease indicates the therapy is effective and no significant change or increase indicates the therapy is not effective. This embodiment may also be used to monitor whether a cancer is returning following surgery with curative intent. The change in cancer DNA over time may be determined using point estimates, confidence intervals or both, and wherein no detectable cancer DNA indicates the cancer is not returning, and a significant change or increase indicates the cancer is likely to be increasing. In these cases, a change of at least two-fold, at least four-fold, at least six-fold, at least eight-fold or at least ten-fold may be considered significant. In these cases, a change of at least 20%, at least 30%, at least 50%, at least 70% or at least 90% may be considered significant. In some embodiments a change is considered significant if the change is greater than a threshold such as 50% and the confidence intervals when quantifying cancer DNA for the first and second time point do not overlap. In these embodiments, a significant decrease indicates the therapy is effective and no significant change or increase indicates the therapy is not effective.

In some embodiments, methods of the disclosure may further comprise providing a report indicating whether there is cancer DNA in the sample. In some embodiments, the report may contain a likelihood ratio or score as described above (or another number representing the same), as well as a threshold to which the likelihood ratio can be compared to determine if the test sample contains cancer DNA. If the report indicates there is not cancer DNA in the sample, but the likelihood ratio or score or another number representing the same was close to the threshold, the report may advise scheduling a follow up test soon (e.g. in one, two or 3 months' time) to reassess if the value is now over the threshold for determining if the sample contains cancer DNA. In some embodiments, a report may additionally list approved (e.g., FDA or EMA approved) therapies for treatment of disease or residual disease, e.g., chemotherapies or immunotherapies. This information can help in diagnosing a disease (e.g., whether the patient has MRD) and/or the treatment decisions made by a physician.

In some embodiments, a sample may be collected from a patient at a first location, e.g., in a clinical setting such as in a hospital or at a doctor's office, and the sample may be forwarded to a second location, e.g., a laboratory where it is processed and the above-described method is performed to generate a report. A “report” as described herein, is an electronic or tangible document which includes report elements that provide test results that may indicate the presence and/or quantity of cancer DNA in the sample. Once generated, the report may be forwarded to another location (which may be the same location as the first location), where it may be interpreted by a health professional (e.g., a clinician, a laboratory technician, or a physician such as an oncologist, surgeon, pathologist or virologist), as part of a clinical decision.

The patient whose sample(s) are analyzed in this method may have any type of cancer or may have previously undergone treatment for any type of cancer. For example, the patient may have or may have had melanoma, carcinoma, lymphoma, sarcoma, or glioma. For example, the cancer may be melanoma, lung cancer (e.g., non-small cell lung cancer), breast cancer, head and neck cancer, bladder cancer, Merkel cell cancer, cervical cancer, hepatocellular cancer, gastric cancer, cutaneous squamous cell cancer, classic Hodgkin lymphoma, B-cell lymphoma, colorectal carcinoma, pancreatic carcinoma, gastric or breast carcinoma, among many others, including other solid tumors and blood cancers. In some embodiments the cancer is a cancer type which, on average, displays an average mutation rate of at least 0.1 mutations per megabase, or at least 0.2 mutations per megabase, or at least 0.5 mutations per megabase, or at least 1 mutation per megabase, or at least 10 mutations per megabase. In some embodiments, the cancer is a cancer that displays an average mutation rate of at least 0.5 mutations per megabase. Methods for calculating mutation rate are known in the art (for example Schumacher TN, Schreiber RD. Neoantigens in cancer immunotherapy. Science. 2015; 348(6230):69-74, hereby incorporated by reference in its entirety).

In some embodiments, the method may be used to guide treatment decisions. In some embodiments, the method may be used to determine if a patient should be treated again, e.g., with the same therapy or a second therapy. For example, if the patient has previously been treated with a first cancer therapy and the patient has been identified as having MRD using the present method, then the patient may be treated with a second cancer therapy that is the same as or different to the first cancer therapy. For example, if the patient has previously been treated with surgery or an immune checkpoint inhibitor and the patient is identified as having MRD, then the patient may be treated with further surgery, the same or a different immune checkpoint inhibitor or another type of therapy, where immune checkpoint therapy includes administration of CTLA-4, PD1, PD-L1, TIM-3, VISTA, LAG-3, IDO or KIR checkpoint inhibitors, and the other types of therapy include, for example, (a) anthracycline therapy (e.g., by administering daunomycin, doxorubicin, or mitoxantrone), (b) alkylating agent therapy (e.g., by administering mechlorethane, cyclophosphamide, ifosfamide, melphalan, cisplatin, carboplatin, nitrosourea, dacarbazine and procarbazine or busulfan), (c) topoisomerase II inhibitor therapy (e.g., by administering etoposide or teniposide), (d) bleomycin therapy, (e) anti-metabolite therapy (e.g., by administering methotrexate, 5-fluorocil, cytarabine, 6-mercaptopurine or 6-thioguanine), (f) vinca alkyloid therapy (e.g., by administering vincrisene or vinblastine), (g) steroid therapy (e.g., by administering prednisone or dexamethasone and (h) radiation treatment, etc. Alternative therapies include targeted therapies and non-targeted chemotherapies, where targeted therapy includes treatment with erlotinib (Tarceva), afatinib (Gilotrif), gefitinib (Iressa) or osimertinib (Tagrisso) which may be administered to patients having an activating mutation in EGFR, crizotinib (Xalkori), ceritinib (Zykadia), alectinib (Alecensa) or brigatinib (Alunbrig) which may be administered to patients having an ALK fusion, crizotinib (Xalkori), entrectinib (RXDX-101), lorlatinib (PF-06463922), crizotinib (Xalkori), entrectinib (RXDX-101), lorlatinib (PF-06463922), ropotrectinib (TPX-0005), DS-6051b, ceritinib, ensartinib or cabozantinib which may be administered to patients having an ROS1 fusion, or dabrafenib (Tafinlar) or trametinib (Mekinist) which may be administered to patients having an activating mutation in BRAF. Many other actionable mutations are known. If the patient is going to be switched to a non-targeted chemotherapy, the therapy may be, for example, a platinum-based doublet chemotherapy (in which the platinum-based doublet chemotherapy may comprise a platinum-based agent selected from cisplatin (CDDP), carboplatin (CBDCA), and nedaplatin (CDGP)) and one third-generation agent (selected from docetaxel (DTX), paclitaxel (PTX), vinorelbine (VNR), gemcitabine (GEM), irinotecan (CPT-11), pemetrexed (PEM), and tegafur gimeracil oteracil (S1)).

Methods of diagnosing cancer are described herein and comprise performing, on a test sample obtained from a patient, a method of detecting cancer DNA in a test sample according to any method disclosed herein.

Methods of treatment of cancer in a patient are described herein and comprise determining the presence or absence of cancer DNA detected in a test sample from the patient according to any method described herein, and administering a cancer therapy or treatment to the patient, or recommending administration of a cancer therapy or treatment to the patient. The administration or recommendation is based on the identification of cancer DNA in the test sample. For example, if cancer DNA is detected, then a therapy or treatment may be administered or recommended.

Methods of treating cancer in a patient are described herein, wherein the patient has been diagnosed as having or is suspected of having cancer based on the presence or absence of cancer DNA detected in a test sample from the patient as determined according to any method disclosed herein. The method comprises administering a cancer therapy or treatment to the patient based on the identification of cancer DNA detected in a test sample obtained from the patient. In some embodiments, the method alternatively comprises recommending a cancer therapy or treatment to the patient based on the identification of cancer DNA being present in a sample obtained from the patient.

Methods of determining the effectiveness of a cancer treatment or therapy are described herein, and comprise administering the cancer treatment or therapy to a patient, obtaining a test sample from the patient, and determining the presence, absence or amount of cancer DNA in the test sample according to any method disclosed herein. In some embodiments, the method may comprise a step of obtaining a test sample from the patient prior to the administration of the cancer treatment or therapy, and comparing the presence, absence or amount of cancer DNA in the test sample obtained before administration of the cancer therapy or treatment with the presence, absence or amount of cancer DNA in the test sample obtained after administration of the cancer therapy or treatment. A difference may be indicative of the effectiveness of the cancer therapy or treatment. For example, an increase in the amount of cancer DNA may indicate the cancer therapy or treatment is not effective. Therefore, the method may comprise administering an alternative and/or additional cancer therapy or treatment to the patient or recommending an alternative and/or additional cancer therapy or treatment for the patient. Conversely, a reduction or disappearance (that is the apparent disappearance, i.e. below the level of detection of the method) of cancer DNA in the test sample may indicate the cancer therapy or treatment is effective. Therefore, the method may comprise continuing or ceasing the administration of the cancer therapy or treatment to the patient, or recommending the cancer therapy or treatment is continued or ceased. In some embodiments, the method may comprise monitoring the effect of a cancer therapy or treatment by performing the methods of cancer DNA detection using patient test sample taken from at least two time points during administration of a cancer therapy or treatment, for example test samples obtained over the course over one or more days, months or years or other time point disclosed herein.

The present disclosure also provides methods of detecting or monitoring minimal residual disease (MRD), comprising obtaining or having obtained a test sample from a patient that has undergone a cancer therapy or treatment, performing a method of detecting cancer DNA in the test sample according to a method disclosed herein.

Recommendations regarding treatments or therapies may be achieved in any suitable way, for example providing a report comprising the recommendation.

Cancer therapies or treatments may be any suitable therapies. For example, the cancer treatment or therapy may be resection of a tumor. The cancer treatment or therapy may be administration of a pharmacological treatment for cancer. In some embodiments, methods of the disclosure may be performed on a patient that has undergone surgery to remove a tumor. In some embodiments, the cancer treatment or therapy that is administered or recommended after detecting the presence or amount of cancer DNA in a test sample obtained from the patient may be a pharmacological cancer therapy or treatment.

Methods described herein may be used to monitor a treatment. For example, in some embodiments methods may comprise analyzing a sample obtained at a first timepoint using the method, and analyzing a sample obtained at a second time point by the method, and comparing the results, i.e., determining whether there is cancer DNA in the sample or determining if there is a change in the amount of cancer DNA or a range of likely amounts of cancer DNA between the first and second time points. In some embodiments, such a change may be determined using point estimates or confidence intervals and a significant decrease may indicate the therapy is effective whilst no significant decrease or an increase may indicate the therapy is not effective. The first and second timepoints may be before and after a treatment, or two or more timepoints after treatment. For example, by comparing results obtained from one timepoint to another, the method may be used to determine if the previously identified variations are no longer present, have been reduced, or have increased in the subject during the course of a treatment. The period between the first and second timepoints may be at least one month, at least 6 months or at least one year and in some cases a patient may be tested periodically, e.g., every three months, every six months or every year for several years, e.g., 5 years or more. In another embodiment, the method may be used to evaluate the effectiveness of a treatment by monitoring patient ctDNA levels at several time intervals following treatment administration. For example, if a treatment is effective, ctDNA levels should rise shortly after administration due to cancer cell apoptosis, followed by a significant decrease as the ctDNA degrades. In such embodiments, the time period between the treatment administration and the first time point may be, e.g., at least 15 minutes, at least 30 minutes, at least 45 minutes, and at least one hour. In such embodiments, the time period between the first and second time points may be, e.g., every 15 minutes, every 30 minutes, every 45 minutes, every hour, every two hours, or ever hour for several hours, e.g., 8 hours or more.

Methods according to the disclosure may also be used to determine if a subject is disease-free, or whether a disease is recurring. As noted above, methods may be used for the analysis of minimal residual disease and recurrence detection. In these embodiments, the primer pairs used in the method may be designed to amplify sequences that contain genetic variations that have been previously identified in a patient's cancer through either sequencing cancer material, cfDNA at an earlier time point, or sequencing another suitable sample.

In some embodiments when testing for minimal residual disease or recurrence detection, the test sample of DNA from a patient would be cell-free DNA. This cell-free DNA may be taken from a patient at any point after treatment. In some embodiments this cell free DNA may be taken at a point that any remaining ctDNA from a cancer would have been cleared if the cancer were successfully treated. This time point may depend on factors such as the initial amount of ctDNA and the treatment modalities. For methods where all tumor is removed at once such as surgery time points may be after 1 week, 2 weeks, 3 weeks or 4 weeks following treatment with curative intent. Where a treatment may more gradually remove the cancer, these time points may be longer such as 1 month or 2 months.

In some embodiments, the method may be employed in a clinical trial. For example, methods described herein may be potentially used to identify a specific group of patients for clinical enrollment or evaluate the efficacy of a new drug (e.g., a neoadjuvant therapy or adjuvant therapy that may be non-specific or targeted to a patient's cancer, or any combination therapy). In some embodiments, the amount of ctDNA in a patient's bloodstream could be estimated at multiple time points thereby allowing to alter the dose of a drug administered to a patient mid-trial, for example. In some embodiments, the amount of ctDNA in a patient's bloodstream could be estimated at multiple time points during a clinical trial and used to determine if a particular therapy, level of treatment, duration of treatment or combination of treatment type and patient is working.

As would be readily appreciated, many steps described herein, e.g., sequence processing and the generation of a report indicating a presence of cancer DNA in a test sample of DNA may be implemented on a computer. As such, in some embodiments, the method may comprise executing an algorithm that calculates the likelihood of whether a patient has cancer DNA present in a test sample of DNA taken from a patient based on the analysis of the sequence reads, and outputting the likelihood. In some embodiments, this method may comprise inputting the sequences into a computer and executing an algorithm that can calculate the likelihood using the input measurements.

As would be apparent, the computational steps described may be computer-implemented and, as such, instructions for performing the steps may be set forth as programming that may be recorded in a suitable physical computer readable storage medium. The sequencing reads may be analyzed computationally.

The methods disclosed herein may be computer implemented methods, i.e., methods that are performed by or carried out on a computer. The present disclosure also provide a computer-readable storage medium or media storing instructions for performing the methods disclosed herein. The computer-readable storage medium or media may be such that, when executed on a computing device, implement methods as described above. The present disclosure also provides a system comprising the one or more computer readable media, a memory for storing instructions to perform the method and the data units (the data units optionally comprising the one or more error probability distribution models) and a processor for executing the instructions.

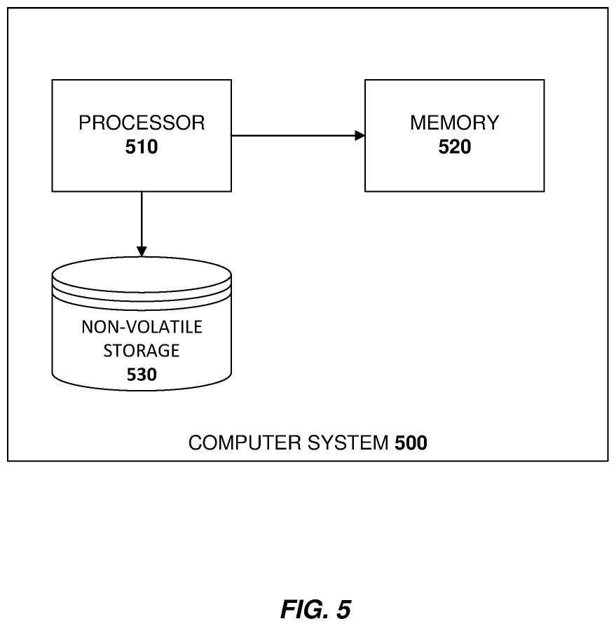

An illustrative implementation of a computer system 500 that may be used in connection with any of the embodiments of the disclosure provided herein is shown in FIG. 5. The computer system 500 may include one or more processors 510 and one or more articles of manufacture that comprise non-transitory computer-readable storage media (e.g., memory 520 and one or more non-volatile storage media 530). The processor 510 may control writing data to and reading data from the memory 520 and the non-volatile storage device 530 in any suitable manner, as the aspects of the disclosure provided herein are not limited in this respect. To perform any of the functionality described herein, the processor 510 may execute one or more processor-executable instructions stored in one or more non-transitory computer-readable storage media (e.g., the memory 520), which may serve as non-transitory computer-readable storage media storing processor-executable instructions for execution by the processor 510.

The terms “program” or “software” are used herein in a generic sense to refer to any type of computer code or set of processor-executable instructions that can be employed to program a computer or other processor to implement various aspects of embodiments as discussed above. Additionally, it should be appreciated that according to one aspect, one or more computer programs that when executed perform methods provided herein need not reside on a single computer or processor but may be distributed in a modular fashion among different computers or processors to implement various aspects provided herein.

Processor-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in one or more non-transitory computer-readable storage media in any suitable form. For simplicity of illustration, data structures may be shown to have fields that are related through location in the data structure. Such relationships may likewise be achieved by assigning storage for the fields with locations in a non-transitory computer-readable medium that convey relationship between the fields. However, any suitable mechanism may be used to establish relationships among information in fields of a data structure, including through the use of pointers, tags or other mechanisms that establish relationships among data elements.

Also, various inventive concepts may be embodied as one or more processes, of which examples have been provided including with reference to FIG. 1. The acts performed as part of each process may be ordered in any suitable way. Accordingly, embodiments may be constructed in which acts are performed in an order different than illustrated, which may include performing some acts simultaneously, even though shown as sequential acts in illustrative embodiments.

### EXAMPLES

The following examples are put forth to provide those of ordinary skill in the art with a complete disclosure and description of how to make and use embodiments of the present disclosure and are not intended to limit the scope of what the inventors regard as the disclosure.

### Example 1

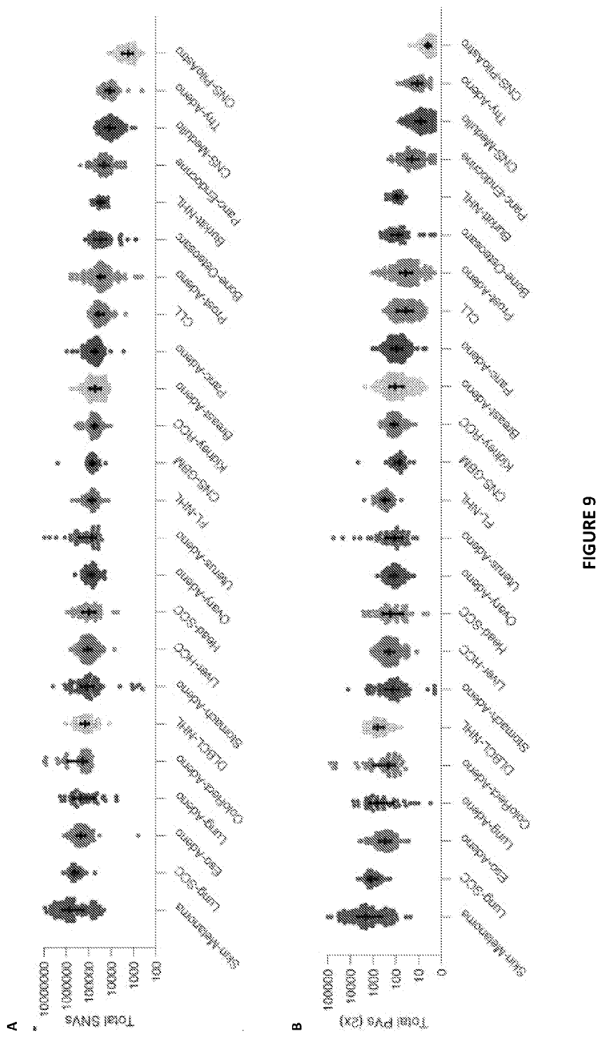

For a tumor-informed assay to best call if cancer DNA is present, it should make best use of all the evidence that can be practically obtained. The typical cancer has many single nucleotide variants present throughout the genome. Often there will be more than 10,000 (see FIG. 9). Individually, these SNVs, if targeted and observed in the test sample when sequencing, provide some evidence of ctDNA, although there is a risk any signal is in fact error such as DNA damage, polymerase error or sequencer error.

When two variants (e.g. 2 SNVs) are close together and on the same strand of DNA (e.g. a molecule of cell free DNA), then, if both variants are seen in a sequencing read or multiple sequencing reads, it provides much more evidence that the sequencing signal is not a false positive (such as DNA damage, polymerase error or sequencer error), and in fact that cancer is present. This is because the chance of getting two errors matching the two exact variants on a single DNA molecule or sequencing read is extremely low.

Whilst these variants on the same DNA molecule provide much more information than any individual SNV, there are far fewer of them. Typically, in many solid cancers there will be 10-1000 of these phased variants (see FIG. 9).

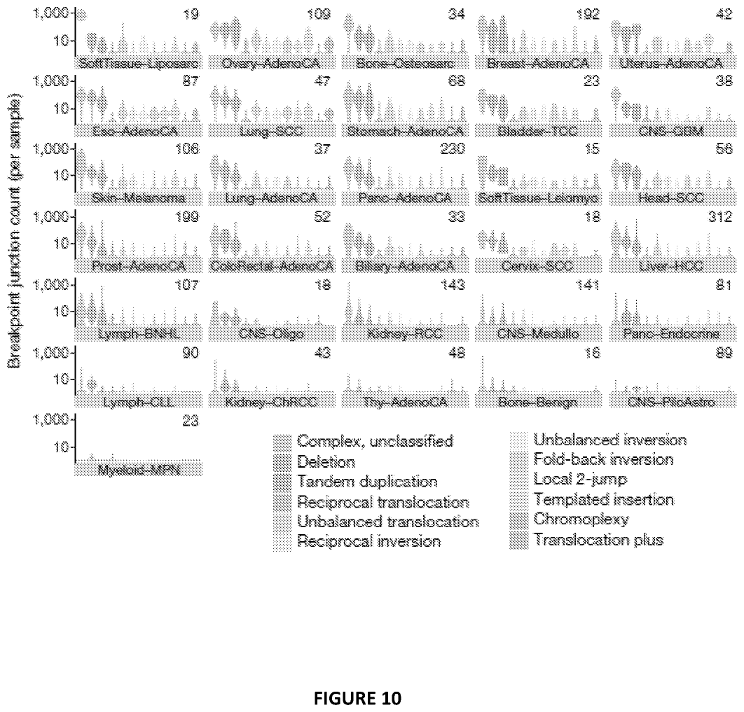

The typical cancer genome is filled with additional changes. This includes germline changes, epigenetic changes and structural variants as examples. Again, as example, some cancers have a large number of structural variants, whilst others have very few (see FIG. 10).

An assay that can leverage all of this information will therefore always have the potential to be better than an assay that for example just looks at individual SNVs or just looks at phased variants. The challenge is how to combine this information. A target region-based approach, where the genome is broken down into sections and then target regions are chosen (the target region being, for example, the sequence between two primers in a PCR product), then each target region is assessed based on all the genetic variants within it, and, when sequenced, all this information is combined together to determine if cancer DNA is present or not.

An assay such as this is also much more universal. An assay that just relies on, for example, phased variants may in some instances have very little information for targeting. As example, for some osteosarcoma patients in FIG. 9, have <10 phased variants. The same patients always have a large number of SNVs however, and osteosarcoma patients also typically have a large number of structural variant (FIG. 10). A target region-based calling approach that combines information would allow consistent high sensitivity cancer DNA detection across patients.

### Example 2

In order to design an optimal MRD assay, the system is designed to interrogate as many high quality regions as is possible. A region might be considered higher quality if for example when cancer is present it is easier to distinguish from noise (e.g. by having 2 phased SNVs) and it is also easier than other regions to amplify and sequence, In order to do this a tumor biopsy is first obtained, it is macro-dissected targeting 50% tumor content, exome capture is performed then the sample is sequenced using an Illumina sequencer. All potential variants are identified using standard Illumina pipelines then given a combined score based on 1) the likelihood of being real, 2) the likelihood of being somatic, 3) the background error rate for the variant, 4) the high signal background error rate, 5) the probability of being clonal, 6) the level of amplification or copy number gain of the variant. The genome is divided into 50 bp windows and these windows overlap by 25 bp. Each window is given a combined score that includes 1) the scores of all variants present within the window, 2) a score for the ability to uniquely align the region (where penalty is given for regions that can't be uniquely aligned and the penalty is higher, the greater the number of misalignments), 3) a score for the ability to amplify and sequence the region (where penalty is given to features know to challenge sequencing including repeats). The regions are then sorted by score and the top 100 are selected for designing PCR primers to. Where two regions that overlap are in the top 100 list, the region with the highest score is maintained and the region with the weaker score is discarded. The 101^{st} region is then added to the list and so on. A multiplex PCR is designed for the top 48 regions. In silico PCR is performed using all primer pairs. When primer combinations are identified producing ≥2 non-specific regions, the primer for the lowest scoring region which is causing this non-specific product is discarded and alternative primers designed. If non overcome the non-specific PCR problem, the region is discarded and the next region is added to the primer design.

One challenge with this tumor informed method of detecting cancer DNA in a test sample is the number of regions that can be robustly and cost effectively targeted. This strategy of ranking regions could maximize the number and quality of regions that are successfully interrogated in the test DNA sample. When the variants are phased variants (PVs), i.e. the variants are in cis, next to each other and on the same chromosome, they can be read together and this increases the ability to separate signal from noise. When the variants are in trans, but still readable with the same primer pairs (or other targeting reagents like baits) the amount of information from the single targeted region could be doubled. The approach should also limit the number of reads wasted on non-specific products.

### Example 3

In order to detect cancer DNA in test samples with high sensitivity, it is advantageous to target multiple regions and multiple region classes. For some cancer types it is sufficient to target just one class of region. However, the inventors have recognized and appreciated that it is better to target multiple classes of target regions containing different kinds of genetic variations. In this example, it is identified that for certain breast cancer patients, a large number of structural variants (SVs) are present, whilst in other patients there are more SNVs and INDELs. Furthermore, many patients have regions with multiple somatic genetic and epigenetic changes. Additionally, they also have many regions with both a somatic and a germline change. A large panel is designed to sequence breast cancer tumor DNA assessing for somatic SNVs, INDELs, and SVs along with germline changes. The optimal target regions are identified in accordance with the methods disclosed herein. Primers are designed to target these regions. Where the target regions contain 1 or more SNVs or INDELs, the primers are designed to flank all the SNVs and indels. Where the target region is identified to contain a rearrangement (e.g., an SV), two different parts of the same chromosome or two different chromosomes will have been brought together. The rearrangement sequence is used for primer design and one primer is 3′ of the rearrangement and one is 5′. In instances where an SNV, INDEL or other genetic variant (e.g., EV) is in cis with the rearrangement, the primers are designed to flank both the rearrangement and other variant(s) using the rearranged sequence obtained from the tumor. Where the target region is identified to contain a pair of phased variants (PVs), the primers are designed to flank the 5′ PV and the 3′ PV. An advantage of this approach is the ability to consistently obtain a large number of regions for assessment of cancer DNA in a test sample.

As each of these different classes are incorporated, different error models may be needed for each type. The inventors have realized that results from such different error models may be combined in a principled manner, e.g. by summing the log likelihoods for each target region class, thus resulting in a high-confidence conclusion of cancer DNA in the test sample.

### Example 4

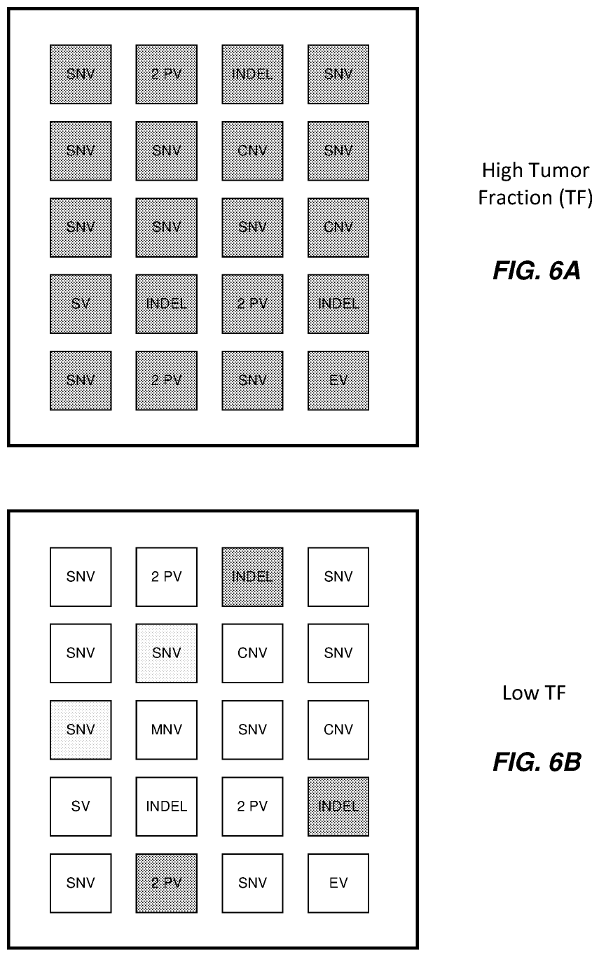

FIGS. 6A-B illustrate why calling a sample as containing cancer DNA can be challenging, particularly for test samples that have a low tumor fraction. As shown in the FIG. 6A (top), in test samples that have a high relative tumor fraction (TF), cancer DNA can be readily called as most if not all target regions will contain multiple cancer DNA molecules resulting in high signal (e.g., a likelihood) across the multiple target regions, thus eliminating most false positives and negatives. As shown in FIG. 6B (bottom), samples that have a low tumor fraction are more difficult to call since the data for individual regions may not be sufficiently distinguishable from the background error rate. Furthermore, the input DNA at such low levels may contain no cancer DNA for some of the target regions, thus for many target regions, once amplified, consequently generate no real signal and are true negatives (see white squares in FIG. 6B). For example, if an assay tested a plurality of SNVs and there was just a single cancer DNA molecule present for some of the SNVs but not others, it may not be possible to call any one region as positive, but by combining information from across the different target regions (i.e. SNVs) it becomes more likely a confident call can be made. In FIG. 6B, 2 SNV containing regions have a single mutant molecule each (see light grey squares in FIG. 6B). In isolation, neither of these SNVs showing small amounts of signal provides enough evidence to make a positive call. Together they provide more evidence, but in this example, they still do not provide enough support to make a confident call for cancer DNA overall. By including not just target regions with single SNVs, but target regions that have multiple phased variants as well as target regions with other variants such as MNVs or INDELs, it is much more likely cancer DNA can be detected when present. In FIG. 6B, there are 3 regions containing 2 PVs. For two of these regions there is no cancer DNA present and no signal (see white squares in FIG. 6B for 2PV). For the 3^{rd} target region containing 2PV, however, there is a single cancer DNA molecule (see dark grey square in FIG. 6B for 2PV). For this, even reading a single cancer molecule can provide a large amount of information as the 2 phased variants are seen on the same sequencing reads, which provides much more evidence (dark grey square in FIG. 6B). When the information from this 2PV target region is combined with the information from the two target regions with individual SNVs present (light grey squares), it can make calling much more sensitive. In this example, the regions with the Indels provide further supportive information (⅔ INDELs are present—dark grey squares vs white square). In order to leverage this information though and confidently determine if cancer DNA is present or not, an approach is needed to combine information from different regions as described in the present invention.

### Example 5

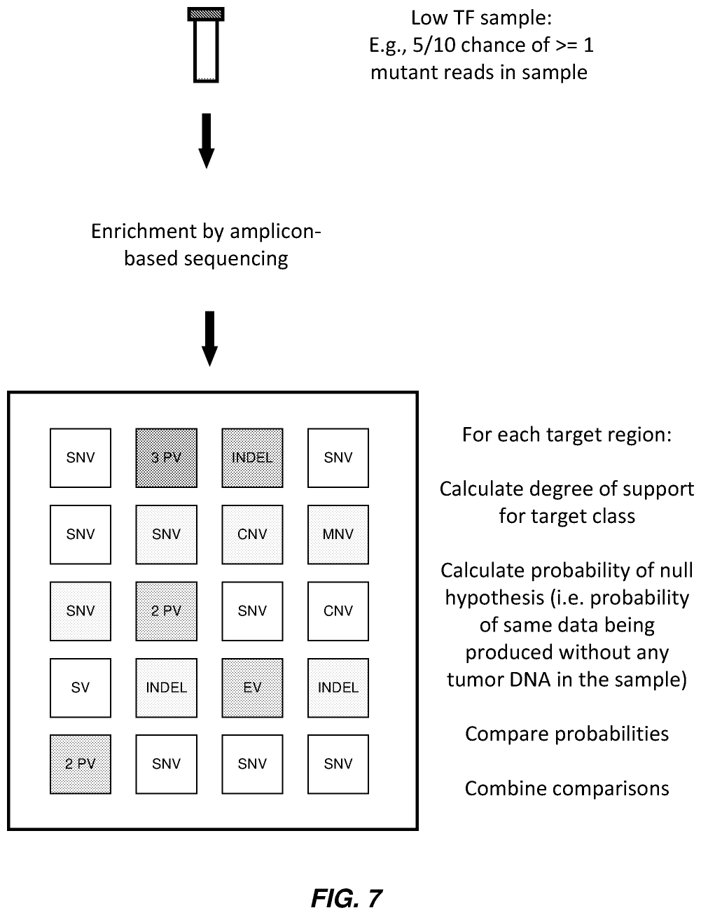

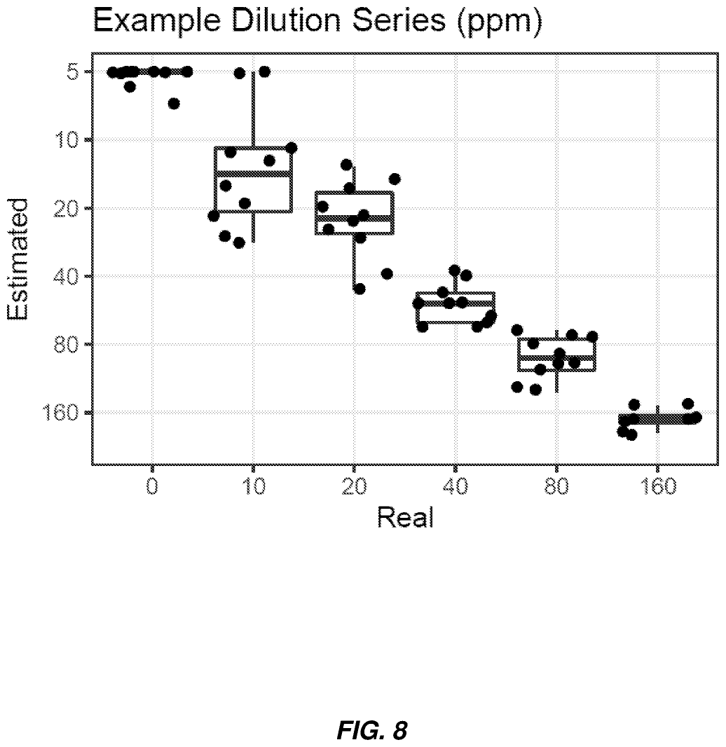

FIG. 7 shows an embodiment of how evidence can be combined across multiple regions. For dilute samples (<<0.1% tumor fraction), the fraction of mutant reads for individual target regions for each sample is not expected to approximate the overall tumor fraction because of dropout effects. For example, many target regions will show zero variant molecules. Instead, the effect of taking n/input reads as a discrete distribution is modeled. In this example, the tumor fraction is not measured directly. Rather, it is marginalized over all possible inputs, which provides an accurate estimate of the tumor fraction of the sample. Specifically, instead of guessing the number of variant-containing cancer DNA molecules across the target regions, the probabilities of all possible values are calculated based on: (i) the number of sequence reads that have the genetic variation or epigenetic variation or combination of multiple expected genetic variations in the target region (which will vary by target region); (ii) the total number of sequencing reads; (iii) the input number of DNA molecules; and (iv) the estimated background error rate for each target region class, and from these, the value with the highest probability is identified. This avoids making assumptions. As shown in FIG. 6, the inclusion of several PV regions (including a 3 PV region) and several INDEL regions provide a large amount of evidence that, when considered together according to any method of the disclosure, can support a high-confidence conclusion of cancer DNA being present in the test sample, e.g. by comparing the quantity of sequence reads for a target region with different error models for each class of target region. The accuracy of the mathematical model(s) can be verified by comparing to real dilution data in a ground truth line diagram (FIG. 8).

Now that the present methods have been described in greater detail, it is to be understood that this disclosure is not limited to particular embodiments described, as such may, of course, vary. It is also to be understood that the terminology used herein is for the purpose of describing particular embodiments only and is not intended to be limiting. Although any methods and materials similar or equivalent to those described herein can also be used in the practice or testing of the present disclosure, the preferred methods and materials have been described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order which is logically possible.

Having described several embodiments of the techniques described herein in detail, various modifications, and improvements will readily occur to those skilled in the art. Such modifications and improvements are intended to be within the spirit and scope of the disclosure. Accordingly, the foregoing description is by way of example only, and is not intended as limiting. The techniques are limited only as defined by the following claims and the equivalents thereto.

1. A method for detecting cancer DNA in a test sample from a patient, the method comprising:
(a) enriching or having enriched the test sample for a plurality of target regions, the plurality of target regions comprising a first target region having a first class and a second target region having a second class;
(b) measuring or having measured the plurality of target regions of step (a) in the enriched test sample;
(c) for each of the first target region and second target region, comparing or having compared the measurements of step (b) that support the presence of the class of the target region to one or more error models which model the probability of observing that class of target region in DNA which does not contain that class of target region;
(d) combining or having combined the comparisons of step (c) for at least the first target region and the second target region; and
(e) identifying or having identified cancer DNA in the test sample based on the combined comparisons of step (d).

2. The method of claim 1, wherein the enriching of step (a) comprises amplifying the plurality of target regions by a polymerase chain reaction (PCR) to produce PCR products, and wherein the measuring of step (b) comprises sequencing the PCR products, or progeny thereof, to generate a plurality of sequence reads.

3. The method of claim 1, wherein the enriching of step (a) comprises contacting the test sample with a pool of oligonucleotides, wherein the pool of oligonucleotides comprises oligonucleotides substantially complementary to the plurality of target regions.

4. The method of any one of claims 1-3, wherein the cancer is a solid tumor, and the plurality of target regions are identified by sequencing the solid tumor

5. The method of claim 4, wherein the sequencing of the solid tumor is performed by targeted sequencing or whole exome sequencing (WES).

6. The method of any one of claims 1-5, wherein:
(i) in step (b), the measuring comprises sequencing the plurality of target regions of step (a) to generate a plurality of sequence reads corresponding to the first target region and the second target region; and
(ii) in step (c), the comparing comprises comparing the quantity of sequence reads that support the presence of the class of the target region to one or more error models that model the probability of observing that class of target region in DNA or RNA that does not contain that class of target region.

7. The method of any one of claims 1-6, wherein in step (c), the), comparing comprises comparing the quantity of sequence reads that do not support the presence of the target region to one or more error models that model the probability of observing that class of target region in DNA or RNA that does not contain that class of target region.

8. The method of any one of claims 1-7, wherein the one or more error models is based on a background error rate for each of the first class of target region and second class of target region.

9. The method of any one of claims 1-8, further comprising training the one or more error models based on a set of control samples.

10. The method of any one of claims 1-9, wherein the one or more error models in step (c) comprises a first error model for the first target region and a second error model for the second target region.

11. The method of any one of claims 1-10, wherein the first error model for the first target region comprises a beta-binomial model and the second error model for the second target region comprises a multivariate beta-binomial distribution.

12. The method of claim 11, wherein the multivariate beta-binomial distribution is a standard Dirichlet distribution or a generalized Dirichlet distribution.

13. The method of any one of claims 1-12, wherein a class of a target region relates to the type of genetic variations within the target region.

14. The method of any one of claims 1-13, wherein the first class of the first target region is a region containing a single genetic variation and the second class of the second target region is a region containing two or more genetic variations.

15. The method of claim 14, wherein the first class of the first target region is a single nucleotide variant (SNV) and the second class of the second target region comprises a first phased variant (PV) and a second PV.

16. The method of claim 14, wherein the single genetic variation is a single nucleotide variant (SNV) and the two or more genetic variations comprise a tumor SNV and a germline SNV.

17. The method of any one of claims 14-16, wherein:
(i) the comparing of step (c) for the first target region comprises comparing the quantity of sequence reads having the single genetic variation and the total quantity of sequence reads for the first target region to the first error model, and
(ii) the comparing of step (c) for the second target region comprises comparing the quantity of sequence reads having the two or more genetic variations and the total quantity of sequence reads for the second target region to the second error model.

18. The method of any one of claims 14-17, wherein the first error model comprises an error probability distribution that models the probability of observing the single genetic variation in DNA that does not contain the single genetic variation, and the second error model comprises an error probability distribution that models the probability of observing the two or more genetic variations in DNA that does not contain the two or more genetic variations.

19. The method of any one of claims 14-18, wherein the two or more genetic variations are positioned within 160 bp of each other.

20. The method of any one of claims 14-19, wherein the two or more genetic variations are separated by at least 1 nucleotide.

21. The method of any one of claims 14-20, wherein the one or more error models considers a distance between the two or more genetic variations.

22. The method of any one of claims 15-21, wherein the comparing in step (c) for the second target region comprises comparing the quantity of sequence reads having both the first PV and the second PV (k₁), the quantity of sequence reads having only the first PV (k₂), the quantity of sequence reads having only the second PV (k₃), and the quantity of sequence reads having neither the first nor second PV (k₄) to the one or more error models.

23. The method of any one of claims 1-22, wherein the comparisons of step (c) comprise a likelihood or log likelihood, and wherein the combining of step (d) comprises summing the comparison of step (c) for the first target region and the comparison of step (c) for the second target region.

24. The method of any one of claims 1-23, further comprising calculating a variant allele fraction (VAF) for each of the first and second target regions based on the measurements in step (b).

25. The method of any one of claims 1-24, further comprising step (f) of determining whether there is cancer DNA in the test sample.

26. The method of any one of claims 1-25, further comprising the step of providing a report

27. The method of any one of claims 1-26, further comprising treating the patient based on the identification of cancer DNA in the test sample of step (e) or the determination of step (f).

28. The method of any one of claims 1-26, further comprising:
A. obtaining a second test sample from the patient at a second time point from the test sample;
B. enriching the second test sample for the plurality of target regions;
C. measuring the plurality of target regions of step B. from the enriched second test sample;
D. for each of the first target region and second target region, comparing the measurements of step C. that support the presence of the class of the target region to one or more error models that model the probability of observing that class of target region in DNA that does not contain that class of target region;
E. combining the comparisons of step D. for at least the first target region and the second target region; and
F. identifying cancer DNA in the second test sample based on the combined comparisons of step E.

29. The method of claim 28 wherein the method of steps A. to F. comprise the additional features of any one of claims 2 to 27 as applied to steps A to F.

30. The method of claim 28 or claim 29, further comprising administering a cancer treatment or therapy to the patient prior to obtaining the test sample and determining effectiveness of the cancer treatment or therapy based on the determination of whether there is cancer DNA in the second test sample and/or whether the level of cancer DNA changes.
